SEQUENCE LISTING (9 sequences)

SEQ ID NO: 1 - protein, 518 aa, Homo Sapiens
MDRAPQRQHRASRELLAAKKTHTSQIEVIPCKICGDKSSGIHYGVITCEGCKGFFRRSQRCNAAYSCTRQQNCPIDRTSRNRCQHCRLQKCLALGMSRDAVKFGRMSKKQRDSLHAEVQKQLQQRQQQQQEPVVKTPPAGAQGADTLTYTLGLPDGQLPLGSSPDLPEASACPPGLLKASGSGPSYSNNLAKAGLNGASCHLEYSPERGKAEGRESFYSTGSQLTPDRCGLRFEEHRHPGLGELGQGPDSYGSPSFRSTPEAPYASLTEIEHLVQSVCKSYRETCQLRLEDLLRQRSNIFSREEVTGYQRKSMWEMWERCAHHLTEAIQYVVEFAKRLSGFMELCQNDQIVLLKAGAMEVVLVRMCRAYNADNRTVFFEGKYGGMELFRALGCSELISSIFDFSHSLSALHFSEDEIALYTALVLINAHRPGLQEKRKVEQLQYNLELAFHHHLCKTHRQSILAKLPPKGKLRSLCSQHVERLQIFQHLHPIVVQAAFPPLYKELFSTETESPVGLSK

SEQ ID NO: 2 - DNA, 11161 bp, Lentivirus
agcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcgagatcccgaaccccgcgtttatgaacaaacgacccaacacccgtgcgttttattctgtctttttattgccgtcatagcgcgggttccttccggtattgtctccttccgtgtttcagttagcctcccccatctcccggtcgagaagcttacgcgtgaatttcgacgcggccgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatggtttagttcctcaccttgtcgtattatactatgccgatatactatgccgatgattaattgtcaacaggtggcgaccggtgatccccctgggctgcaggagagaggtcggtgattcggtcaacgagggagccgactgccgacgtgcgctccggaggcttgcagaatgcggaacaccgcgcgggcaggaacagggcccacactaccgccccacaccccgcctcccgcaccgccccttcccggccgctgctctcggcgcgccccgctgagcagccgctattggccacagcccatcgcggtcggcgcgctgccattgctccctggcgctgtccgtctgcgagggtctagtgagacgtgcggcttccgtttgtcacgtccggcacgccgcgaaccgcaaggaaccttcccgacttaggggcggagcaggaagcgtcgccggggggcccacaagggtagcggcgaagatccgggtgacgctgcgaacggacgtgaagaatgtgcgagacccagggtcggcgccgctgcgtttcccggaaccacgcccagagcagccgcgtccctgcgcaaaccgggctgccttggaaaaggcgcaaccccaaccccgtgggaagttcctatactttctagagaataggaacttcgaaacttgatctgtcgccgcaattcaagcttcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttggcctttttgagtttggatcttggttcattctcaagcctcagacagtggttcaaagtttttttcttccatttcaggtgtcgtgaacacgtgctcgaaccagtgaaggccagccgccatggattatccatacgacgtcccagactatgctgggggcgaattcgtcgacggaggaacaagtttgtacaaaaaagctgaacgagaaacgtaaaatgatataaatatcaatatattaaattagattttgcataaaaaacagactacataatactgtaaaacacaacatatccagtcactatggattaggcaccccaggctttacactttatgcttccggctcgtataatgtgtggattttgagttaggatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcactatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgacgaccgatatggccagtgtgccggtctccgttatcggggaagaagtggctgatctcagccaccgcgaaaatgacatcaaaaacgccattaacctgatgttctggggaatataaatgtcaggctccgttatacacagccagtcatagtgactggatatgttgtgttttacagtattatgtagtctgttttttatgcaaaatctaatttaatatattgatatttatatcattttacgtttctcgttcagctttcttgtacaaagtggtgctagctagataactgagctgatctgacatgtcctgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttagcgtgacgtcaattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattgcgcacgcagtgaagttcctatactttctagagaataggaacttcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtttaaacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcgtttaaactcactaaagggaacaaaagctggagctgca

SEQ ID NO: 3 - DNA, 1551 bp, Mus musculus
atggacagggccccacagagacaccaccggacatctcgggagctgctggctgcaaagaagacccacacctcacaaattgaagtgatcccttgcaagatctgtggggacaagtcatctgggatccactacggggttatcacctgtgaggggtgcaagggcttcttccgccgcagccagcagtgtaatgtggcctactcctgcacgcgtcagcagaactgccccattgaccgaaccagccgcaaccgatgccagcattgccgcctgcagaagtgcctggctctgggcatgtcccgagatgctgtcaagtttggccgaatgtccaagaagcagagggacagtctacatgcagaagtgcagaaacaactgcaacagcagcagcaacaggaacaagtggccaagactcctccagctgggagccgcggagcagacacacttacatacactttagggctctcagatgggcagctaccactgggcgcctcacctgacctacccgaggcctctgcttgtccccctggcctcctgagagcctcaggctctggcccaccatattccaataccttggccaaaacagaggtccagggggcctcctgccaccttgagtatagtccagaacgaggcaaagctgaaggcagagacagcatctatagcactgacggccaacttactcttggaagatgtggacttcgttttgaggaaaccaggcatcctgaacttggggaaccagaacagggtccagacagccactgcattcccagtttctgcagtgccccagaggtaccatatgcctctctgacagacatagagtacctggtacagaatgtctgcaagtccttccgagagacatgccagctgcgactggaggaccttctacggcagcgcaccaacctcttttcacgggaggaggtgaccagctaccagaggaagtcaatgtgggagatgtgggagcgctgtgcccaccacctcactgaggccattcagtatgtggtggagtttgccaagcggctttcaggcttcatggagctctgccagaatgaccagatcatactactgaaagcaggagcaatggaagtcgtcctagtcagaatgtgcagggcctacaatgccaacaaccacacagtcttttttgaaggcaaatacggtggtgtggagctgtttcgagccttgggctgcagcgagctcatcagctccatatttgacttttcccacttcctcagcgccctgtgtttttctgaggatgagattgccctctacacggccctggttctcatcaatgccaaccgtcctgggctccaagagaagaggagagtggaacatctgcaatacaatttggaactggctttccatcatcatctctgcaagactcatcgacaaggcctcctagccaagctgccacccaaaggaaaactccggagcctgtgcagccaacatgtggaaaagctgcagatcttccagcacctccaccccatcgtggtccaagccgccttccctccactctataaggaactcttcagcactgatgttgaatcccctgaggggctgtcaaagtga

SEQ ID NO: 4 - protein, 516 aa, Mus musculus
MDRAPQRHHRTSRELLAAKKTHTSQIEVIPCKICGDKSSGIHYGVITCEGCKGFFRRSQQCNVAYSCTRQQNCPIDRTSRNRCQHCRLQKCLALGMSRDAVKFGRMSKKQRDSLHAEVQKQLQQQQQQEQVAKTPPAGSRGADTLTYTLGLSDGQLPLGASPDLPEASACPPGLLRASGSGPPYSNTLAKTEVQGASCHLEYSPERGKAEGRDSIYSTDGQLTLGRCGLRFEETRHPELGEPEQGPDSHCIPSFCSAPEVPYASLTDIEYLVQNVCKSFRETCQLRLEDLLRQRTNLFSREEVTSYQRKSMWEMWERCAHHLTEAIQYVVEFAKRLSGFMELCQNDQIILLKAGAMEVVLVRMCRAYNANNHTVFFEGKYGGVELFRALGCSELISSIFDFSHFLSALCFSEDEIALYTALVLINANRPGLQEKRRVEHLQYNLELAFHHHLCKTHRQGLLAKLPPKGKLRSLCSQHVEKLQIFQHLHPIVVQAAFPPLYKELFSTDVESPEGLSK

SEQ ID NO: 5 - protein, 495 aa, Mus musculus
MRTQIEVIPCKICGDKSSGIHYGVITCEGCKGFFRRSQQCNVAYSCTRQQNCPIDRTSRNRCQHCRLQKCLALGMSRDAVKFGRMSKKQRDSLHAEVQKQLQQQQQQEQVAKTPPAGSRGADTLTYTLGLSDGQLPLGASPDLPEASACPPGLLRASGSGPPYSNTLAKTEVQGASCHLEYSPERGKAEGRDSIYSTDGQLTLGRCGLRFEETRHPELGEPEQGPDSHCIPSFCSAPEVPYASLTDIEYLVQNVCKSFRETCQLRLEDLLRQRTNLFSREEVTSYQRKSMWEMWERCAHHLTEAIQYVVEFAKRLSGFMELCQNDQIILLKAGAMEVVLVRMCRAYNANNHTVFFEGKYGGVELFRALGCSELISSIFDFSHFLSALCFSEDEIALYTALVLINANRPGLQEKRRVEHLQYNLELAFHHHLCKTHRQGLLAKLPPKGKLRSLCSQHVEKLQIFQHLHPIVVQAAFPPLYKELFSTDVESPEGLSK

SEQ ID NO: 6 - DNA, 25802 bp, Homo Sapiens
gccaggtgctcccgccttccaccctccgccctcctccctcccctgggccctgctccctgccctcctgggcagccagggcagccaggacggcaccaagggagctgccccatggacagggccccacagagacagcaccgagcctcacggggtaagaggcccatggccctgcctgcaggtggaagagactgctggaggttcagaggagggcagaaatggcaagagactgggcaagaagtgaggaggggagggagggacaagagtgcagaggagaaagccctgggttggcagggagtgaaactggaggagaaacaggaggaggggctggggagatgacagaggagaagaaatcgcaaaaggagagaaacaggaaagaaagggagaagcgggagaaggaagaaacaggttggagggtagatggggagggaagagccaagtcaggggagccagatctagacccagcgaggggtcagaatgtggacaggccctcccaggtgtgggcttaccccccaggaggcacagggacactgcttccttgatctggccaggaccccagacagcagtggccgggaggctggctgctccaccttctgttcattcaccagtggagaaacctgtcctttaaggagtgacagacttggggagtgtggcagggggaaccacaccctgtcagctgcccatgaccacgtagcctggtcctttcctcgccactcacccagggatgtgggatttcatctctgcagagaatgtggggcactgggccatcagcagggaaggacagaatgtcaggccgtccttggccccacccatggtccaagtcaggaaccccctcagagcaggatcgcagggccacaggcttggtcagctgtgagcatggaggaggaaagtttgagggccagagccaccaagctccagggccacctggtggaggaagaactggctgcctgggtgagcaaggggagcacccccaccacaggactgctctgaggtgcaaggctagaggccaggagccggtgccacgtcagggaggaagctgggtggggccaggcaggagcaaggggaggtgggcggggctgggtggggccttgccacctcggggcagtcttttctcctccctcaccccagcagagcccaggccatcaggccactcgcctccacacagcctagtgaccacggcctgccagtggggagcatctcccgggcagaagggcactgtgtgtgtgtgtgtgtgtgtgtgtgtgtgtgtgtgtgtgtatgtcaggtgtcaagactgaacagcagaaggaaaaaaataacagaatagagtcagaggttagggaagctatagggggtatagggcatggttgaagggctttctgagacaaaagactggccatgtgttctgtgatgagctgctatgtggatgtaggaaatgggccaaggtctggcaggggaatcctctttccaggggctccctggctaggtgtttcatagatgttaactgcatgaatgggtgacacccccccacccaccaaccatgctccagctaaggccctcccctcaacggctgctccccctggaggtccgagtatccatgtgacccccatcgacccactcatgctccctactgcccaatctccacccagacccggccacgcagcagcagcagagaagggaggccttcctcttcaggctggtaggccagggaagcctggcccgtggaagccagagaggggtctgtatttgcaactctcccctcctcttcctccaaacatcctctccccctactcccaacccccacatctctgaagccctggtgttttcaggcatagataagcaaccaagagatttcagtcccacagagatttaactctggctgtaggatatttcttgtgcacctccaatgaaattacaaagtaaataagaccaacacagtccctgccttcacagggctttacaatttggcaggaaagacccatttcatagacaatgacacaattacagcagttaggtgacagttggtgacatggtgagtgccaggaaataaaatgcaaggagctaaatgtgtcagcacaagggcaggggaagggggtgtgtgtggcttctccaaggaagaaacataatcagagacctgagggagaaagagaagttggctagattaaggggtacaggagggacaggcattctaggcaaaggagcagcttgggggagtcccagaggcagtgaggagcctgacacttctgggtacctggaagagggccagtgtggcccaagggcagaggaggagagaggggcactgcagggggtggcctgcctgcccttcagacgccaggctctgagggaaggagtgcggcagtgggatcctaggagtggggaaagtgtctcagcccctcatgatcttcaacctttgacgtcaaccccctctcttgtctttacagagctgctggctgcaaagaagacccacacctgtgagtagggggtccggggcaagaaaatggctggggcataatgagatggagaataattcagcaagtgatctgtggggggactgaggttggacagaggtggaggagtcaggaagagatggcaggcaacaggaaagcaaggggaggtgcgaggaaaccacagatcagggcagtgggaggagatgaggaagaagcctgaagcatgaaaggaaacactgtaaagaaaccaggagagaacaaaggaaaagggaaccgccttgtgaaaaaaggcgaggagaaatgctggacagaggcagggagatgggagggggtggggccgactgccaggccggggaaggaaggagatagaggtaggggggtgtgcggaggcggggtggcctcaggaactcaggaactcagcgctgctgccaagggagacagggagcctgcaccaggctttttttttttttttttgagacagagtctcacactgtcgcctgggcgggagtacgatggcacaatctcggctcactgcaacctccacctcccaggttcaagcgattctcctgcctcagcctctcgagtagctgggactacaggcacccaccaccacgcctggctaatttttgtatttttagtagagacggggtttcaccatgttggtcaggctggtctcagactcttgaccttgtgatccacccctctcggcctcccaaagtgctgagattacaggcatgggccactgtgcccagcctgcaccaagctctgtggtcctgagccaggtttggtgttcatctctgtggttaacacatcctgaggtgtgaacaagcctggagccagcctgtgcaggactagagagcaggagagctttatctctgtaggaaacagaatggacaggggcactctgtgtcctggggccctgaaagccagactacagcaggtaagaagggcactagagctcagagaggacttcccagtggacgctagtgaataagaatcctctctagggaccactcagcacatcagctctcctctcctagtttgtggcaaaggctagtcccccagagaatgcacacagtcccaaagctatgcccatcagggccatgcgtgcccttctgtgagcatgggtccctgaatcgagaggatccctccagggtaggagtggccacaccatttctccatagccgtcccttgactgccttgccagttcctcctgagagctcactgccatctcgcccaatgccaaaattgtctagagcctccctgttccaagtagagacatctcagagagcaaagcattgcccttctccatgagctgggcagcccagggctgagggccatccaggatgctacctcctcctgggtgggggttgctttgctcagcataatcctgatcagtgtctcgcctgcccctagccacagggccccacggaaatgctgagcagggccccgggaggaaagcgggtcagggagtgacgcgggtgagctgggggaggggggtcacggaccttatgtgggtggggggagtagggggactttcggagcagcaggaactgggagaggtcaaaccctattcctacccttaacccctgacctcagaaacctcagccaggactatacctccttatccctttcccatcccaccagtcctcgtgtccccaccccaagagtgttgtcctgaggatccaggctgacagcctctcccccactccacggcccagctcctgaagccccaggaaggcccaggaaggcaaggaaaggcctgtgcctggatgagagggtctggagtcccagcaagatcagacacctctcaggagtttgccccacagaatccaggagacattaaggcctcccccaggccgagagactctgaaaattggctcctgaattgagacaaccccaagatgggggtccaagatggggaacccgggggaactccccagaccaagagggagcctgaatactgaggggccctgaagagggactctcaaagccaatgtaccccacaaagcacctaagaggctgagaaggtctctggaattgtttggcccctgagaaggacccccctcccccaggccaaaaaacactccagagagactcacataccgagggacctcccaggctgagacaccctcaaaagcagacttgaaccagagaaaccctcccatccttcaagtgggatccgaagccagtctgcctgggcttgaatcctgactctgccacttactagctgtgtaaccttgggcaagttacttaacctctctgtgtctcactttcctcctcgaaatgaggataatggtagctacttcataaagtttttttattttttctttttgagacagagtcttactcagttgctcaggctgtagtgcagtggcattatctcagctcactgcaaactccgcctcccgggttcaagcgattctcctgcctcagccccctgagtagctgggactacaggtgctcgccaccatgcctggctaatttttgtatttttagtagagatggggtttcaccatgttggccaggccggtctcgaactcctaacctcaggtgatccacccaccttgggctcccaaagtgctgggattacaggcgtgagccactgcccccggcccacataaagtattttgtgaggattagattatccacataaaacatcgagaaccatttctgacacataatagtaagcactattattatgattatgagtatgatgatggtgatgatgatcattatctacactccaatttcagcagtttggctcctaaggaaatttctggtttccttctgtggattgtgggtatttgcctggtgattattactgcttctatcatttccatgtattccctagcgcaagatatatgtggtgtctgcagggaggagcgtgggcatgggagtggtgggacagccccccaggctgcaccacactgggtatggctgttggcagtccacagggtgtcaggtggcacatgccaccaccccaggcagacatcctgtgacttacctctgaaaacatccctgtccctgaagtcacctggcaaagagggtcaggcttggaaggtcctcgggggtaggaggagtagaaatttggcttcaggctcgtgtgagctccattctggctccccagccagaactaccctggcgggtggaaacagcttttaccgcgtgtggctgtcgcatgtggttttggaattttccaacgccccctacgattggctgcccctcccctcacaccctgccccaggcccagattggccacgtggggcgcctgtcatcctactcactgcaccccttgggggtggggtggggggttgtcacttggccacctgtgtggtgcagagcttaaaccccccagtccagaagcactgggggagagagctaggtgcagagcttcaggctgaggcgctgctgagagggcctcgccccgcctctgccgccagctgcaccccactcctggaccaccccctgctgagaaggacagggagccaaggccggcagagccaaggctcagtcatgagaagtaagtgaatggggccacctgggggcgggggagcctggaccctgtcgtcacccctctggaaaggaggatgtcttgggaacagtgtgagcaaatctgagcaaagcagggtgcgtcctggtcaaggtcaagatctgttgggagatgggtggagcagctggggagtcctgctggccaggcggctccctgacagcagcaatggtaagaggactgttgaggaattcctgagttccaaaaagaacctccaaaagtatctgaggtatccaggcccttaattttacaaaaagactgagagccaaggaggccgcgtacttggcccaatgttacacagctggtatctgatgggggctggtaattactaggggccctgaacgcactctcctgttacagtgtttcccttgttacggtgtttccctctgtgcctccagcctgcttgtgagtaaaaggaggccagagcagttgctgtgctctgaagcaacaggacagcgagagtcaactgaggttggcaggcagcagggcacagaatccccgcgtccaggcctcaaagccaccaggctttctgcccaatccagggacatgaggactatgtggaatccagtcagggtgcacccgggcacatgcctgcgtgtgtgtgcatgtgtgtgtgtgcgtgtgtgtgtggggtgtgtgtgtatgtgtgtgtgtggttctccagggtcagtcacccatttgaccggggaagcaaggcaatggagatgggattggaaagacctcggcctcacccttttcctggcttggagccctgggccaacagttggtgggcagatgtctggggttcctgtgccttccagcagagcaagggtgggcaacctcaggaagtcccctgtatttgttgtgaagggaagccagggaagccagagaagctctcagagcacaaggtgaaggaggaaaagggaagccccatgcccctgtggctgaggcctgaagcagccccagcttcaacctggggtcttcccacaagccacagaagggaggccgtattgagtcctggaaagacccctggactgacagtgtaagaccaggcacttgtgccgcctatcactggctagccatatagacttagacaagccaagtcactgggccctggtctaccatccgcaaaacaagggagctgaattttaagatagaacaacaaaaccaactatggagagtctcccattccaaaggcccagggccaagagagagcccagtgccctaaggcaggcatccccaagggtgatgatggagactcagctctcccagaggttagaaggaagggaaaccaaggaaagaaaagaattccacttctgtgacagggactgaggccacagtgcaggcatatgagctggggtggataaggagggctggagagaagagccaacagcttctacacagaggaaagggccccttggagcttccgtttcccttctagcttctctctattagcagcaaacgctggcagactggtcttcccagagatggcccctaatgagcaaatcaaccctgggatgaaatgactaggtgtgcttctagcccagcagcggggcaggagtccccaggaggggctgagggagacacttggccagctctccattattcccacccccaccacacacacacagccccacccaggcggggaagtaggagagagggcctcctccaacgagggggtcagtgccactggtccttctcacttggacatttacaggtatctcatagccccaagttatctgaccccaatgagtgccaaccaccaccagcccctccaaccgccgatggcaaaagtcaaagcacatggggcttgggactgtgacacacccagagaccaagggcccgacaaaaggagcagaaacaaatcaagaaataaggagagatgcagaggcccaactcagagggcgagtttctggaacacctagggagagagtaatgtccagctggaggtgtggttggagaaacaccaatagagccaaacgcatggaggtgcagccgggcgtggtggctcatgcctataatcccacttcaggaggctgaggcttgaggccaggagtttgagaccagcctgggcaacaaagtgagccgctgtccctacaaaaaaagttttttttaattcgctgggcatggtggcgtgtacctgtagtcccagctactcaggagactgaggcaggaggatcgtttgagcccaggagttcaaggctgcagtgagctatgatcaatgtcacagcaccctagcttaggtgacagagcgagaccctgtctcttaaacaaacaaacaaacatgaaggtgtgaagcccagaaacagagctggaatgcagaagatcacaaggcacatagacacagggagaggcggagagaaaagataactggcagtaaagggaagacagggagacacagggtctaggcaagcggtaagagggaagtagggtggctctgacaaggatggcaccagcaccctgccatccgacctgctgctgtcaagtggagataatgtgtctgtacgccaaagagactcccagccagccagacggagggtagcccagacatcttcccctaagggcatggccagcgtggcgccaataggcactcaggccctggcatttggtctggatggggaggtgggaggccgaggggtgtgaggtcactgtctatgagctgatctttcttaggtttggtcatgatcatgaggaacagtgggccagaaaggactccaagtccccagggtacacagatatcaccctgagagcccagggccatcttaggactccgtgaagggcttcttccttcctttcatcactccagcacccttctctttccccgtcttcccattcctaacagaaataccaaaccaccgagcccaaggtgctagactctgaaaagactcagcttgtcccagcacaatgaaaatattcggtccctaccaagggctcctccccatcccctgccgctgtgctgctgactcaaactaacagggaacaggactttccaagggctaaggtctctcccaggcgggcagcccaaggcccgcgctcctgttcccatatagctagtcctttctgaagtctcacctccacgcctgctgctgcagtgcagccccttccctggggctctgaggctcaaaaaggatggagatgtctatttcgggggagggacagtaacaaggcagggcctcagggccagagccaaggtctttctgggtccactaccctcctggagcagtgaaggtgctctgggatgagggacagatggccctgaaggcagggaaggtgctgatggtgacgtctgggctcccactcgccagagcttcctcctagtgattcatcccctcccccattcactggttgttttcactcgcctttctccagtcccagactgtgggggtggcggaggcaccaggagggggtttcgggtggctggctggctgtcattcatggcttttcaaaaccccagactctccctcgcccacctgagttttagcttcactattttctcagccccaggatctgggtgtttcagcagaaaattctcaccaggagctgcggtgagggccctggcctgggctgggggtagtgttcgccaggtagaaccacttctcccccagcccccacccagccctcccgctcctgctcccctgtggtggcacccaccaccttggtttccagctttgcagaagctgggggaactgagagctgagaaaggagggaggctggtgacaggttggggtgtgcaagtgacccctgaccggagctgtggtctcggtggggatcgagacagagagttctggctgtggggagaggaggagctcaagctaaggaaagagatgcattcctctttctcactcattcattcattctgcaaagggaggcataccaagatgtatggaaacctagtctataccctaaaggggctgtggtgttcctgaggagtccccgggaaccctgttgttgatatataagtcatcccacttggccttagagaaaggggtaagggcaggaggaggctgggacctcagaggaagagttgggacagagggaataatatctcttgggatctttatacaaacaggagtcctggctttagttctgtgggtttcctctgtgaattctgttcctagaccaccacaatcacctcacacttttacccctacagccaaataaccatgtcctctacccaggtctccggtgtgcatttgggcgtcttatcctcataaagtatatagcttaaggcttgagggcactggctgcagaattacagagatctaggttcaaatgccagatctgccacttctggctgtgtgaccttagtcaagctgtttattctctctaggcctcagttttctcatctgtaagatgaggatgatactagcagggcctatttcacagggtggttgtgatcattgaatgaaaatgcatgtaaagcctggcacagagtaagaattcagccaatgcttatgtctatcattatcctttctccttaatcagtatccactggagaaaaaggagagagaaagaggagaggaaaaggcccagctctagattgacgaggcaccaggaggagatgaggaccccggggtttctctgtgtgaacccattctctaccatgatggtgggggggtggtggttctggggtttgagaggagagatccccctgggttgcagcccctcccctgctcctaccacttcctctctgtggcatagggcacctggacagggcccagggttggcttcctaagctggcacagggccaggctctggaagtactcaaagcactatcattgaatctcacagctgtgagaggctcgattagaggaggatggaggactttgcctctaccgtatttcccctcataggaggagctactaggatgctcctgggggtacttcagtctctaaagcaattcagaggagattcagtttctttttctagtattgtctcagaatgaagtcttagaggtcacactggatggcagaaaagatgcaagaatgagctcaggaaaagatcgccaaccccagccccactccctggaaggagcagcctggcaaaaaggcaggaatagggactaagttcttgctactcaaagtataagctgaagaccagtaagcttaggcaccaactaagagctgaccaaagtgcagaatcttgagtcccacctcagatccccaggtaatcttgtttgagatgcagaaagccatagtcattcagtcattagatgacttaaggagctggcgggtgcatttgggcatttgtctctccttttggaagatgtggaattggagttttcttttggagaaagctctctctcaggagagagctacagtgagagagctgcaggtgagatgcaaagatctttccaagacagcaggcgagggtagagttagggctgccatgggagcagtggtgcaggggctggttcgaggttcctgacctttgcttggctctagttcctggcactggcaggcccttagatggtaagttcctggcaggcctggctccagggcaacagtttgagtaatcctgtgattactcagctccttctccccaatctgagttcccaccacctgctgcttcagggacgtgtctcccaggggtagcttttgaaggtgagttatcatcagaggtgttttcagctcctctcaccaacccccaagtctaccagcccccttcccgttctcccccaccccttctctacaagtaacacacaccctcatgtacacagcactggtactttctttctgaggtttgggtggatggcattgaagggagtgcaggtggcaggggaaggaggggggagcaggactagagggcctgaagtgctgagggggccatgggttgggcaaaggccaggatacaagcgtggattttctgtgtttcagtgtttccgcagccgtctctccatccctagatctatgtctctgtttttcctggacccaaacctcttggtctctccagctcagtgaggctagggggtctgacattctagctcagagtctttggccttgtgtgtctgtctcactgggtacttctgtcttggactgggaggatggaggatgggctgaccaggtggctggagtccaaggctttggtgtcgcccaaacatgggtatttgcgaagggggtccttgactggccgcatcttcctatgagaaggtcaaggtcttggtgacactgttgtgaggttcctagggccaggagaagcagtgggtcaagcatggggcagggagatgtgtctggggtcaccatgtggtcagcacacacacgcacgcggatgtggctcttggagaagggaagaggcagggaggcctgtggttgactgctgggccagagcccgagttcccactgcggcttggcctaatctccctcccgcttacttttttgggtggcccacgttggtttctcccatctctggtcttaggccactgaaaagagagatgccacagtgttggagggggaggtgttgggcaagaacagagtccacagggcccgagtgctcctctcagccttgccaggcccactggtcttctcagcctgacctctgctcaggagtaacttgaatacacagtgctttccattggtgatatcatttcattactctctgttacctacaatcaccccatggacaggcaataatacttttatcctgatcttgctgacaagaaaacgtaggtctagagggagaatctaagtgactagaagtatgggggccccaacgttttctgcccctgttccagacacccagtgccctcttattctctcttcccctccatgtattaaaatctgctcttaatttactggaaagaacctggaaaactcacttaccgcctggtcctggggggaccacagcttacagtgtgagcactggaagcttgctgctgggcaggcacgtgtggctgggggttgggtgaggaattgagtgctaatgaagggatttcagatacccatgtgccctgtccccgtcagggtcagctactgcctctgactcacctcaagtctggcctgagggatgatggtttaagaatgatggaaataatattttggggaatggcgggcaagccatggtctccctgcccacttcacatcaacaggccctacatgtgaccagtgtccaggccagggactggaggtgagatcacagcaatgtgcattcgccctccagagaggctgccacttcttttgcccctttggtgggagacacgtcatagattaaaaggggctttggcccttctcttcacttttctggcctgtcccgccccagggctccccactcagaggggaggcagcaggtcagtgatggtggaaaagagacccatgtgctctgggaaatgctaaaaacacctacagtaaccagggatcccaagctttgcccagctacctttcaaggaagagctgggagaaggcctcctgtggtcccatgaagctcagcagagagtcgtgggcattgacaggaacagggcaaagcagagatgagcttagggccagcagcaaaccacaggggtagggaagcaggtacaggttacagtgagttcagtgggagagagaacccggggtcctagtgccccatgagcagacactaacatacaaatatgcacgtgtggacgcaggagcaaccagatatgtcgtaggctcacaaatgacacatacttttcttgcatatgcacacccacaagcacaccttcaaacatgcccactgaaatgtgtacccgaaaagacaggtacacagaacagcataatggaaagctcaggggaactgggatgtagatccagctctcaggcaaactagcctatgaccctggcccaaccgctttgcctcatctggcttagtccttcatcggtaaatgaagaggttgaatgagatggtctctagtctcagccatttttaacattgatgattcatgttcacacaaatggatacagatgcctttgtacaaacataagtgttcacactcagacacacggatgtacacaaagccaccctctctctgctctggggccaagagcctaagagccctggctaattctttccctaggctctcaggcatccagcagagctggggtgttgagcccccgtttcctgggttcctccctgccaccccctcaccctgtctctgtatagcacctccctgagccttcactgtctggctgggaaggactggcatctctgcctatcccccaccccttgtgtaccacatcttcctgctataccctacactttgcccatgggagctgagccccagcgagggagggaggcacagaggaagccccttcggcgggaagcaggtgttgtgaggccgtgaggagtcctatgtcccaaggcgggagggaggcaactggagcttttcaactcgaagggctgagcaggtggcccctctgcagctgctttctctgcctccatagcactgatacaactcccggccacccctccacactcccctcctctgtgaaacaaacacagcttcctcacaccctttgctgagaagcatttggcatttcacttccccttccattttgcaagagggaaacagcaagctggagctggttggagaagtagtggaagcaccaggtacctgtatctgcagcccaggtacatggtggttgaatgcagaaaagcctcctgggctgacctacttctctttctctcttccagcacaaattgaagtgatcccttgcaaaatctgtggggacaagtcgtctgggatccactacggggttatcacctgtgaggggtgcaaggtgagtcataggcatgtgtatgcatgcatgtgtgcgtgtgcatacacaagcgcgcgcgcgcgcacacacacacacacacacagtgtctccttagagataaacaagggggttaatggcctttgttctgactccagggatgatctcctgggcagccaggaaaatgcctgagtagcgccttcctgcagggccctcaacactggcagggccctgtcttaagctggggaaatgactacaggataaattgcaattacacaaatagatggaggagagagaaaactgacaggtcgtgggatgtagaaaagctgccagagcttgtgggctggaggcctttgtgagtgagctgggcctgaccaggataagcagtcttgccctccacctgctttccccagggcttcttccgccggagccagcgctgtaacgcggcctactcctgcacccgtcagcagaactgccccatcgaccgcaccagccgaaaccgatgccagcactgccgcctgcagaaatgcctggcgctgggcatgtcccgagatggtgaggccaagtcgacagccccctggggttttcctggtgtctccagaggggcagcctggcctgctgagctagacaaggcttaacctgcaagacggcccatcctctggtctcctctccattcctccccgttacaaccccttgtctcctcccctccagggatggatgggtcacccccatcaaagttctttggagtccccaactttaggctttgctagaacaaacatcctctttccaaggtcccatacctcttcatctcttcttacttggaacgaattctcctcactctgctgccctctggcccagctcccatgcagctctggcaccttcccctgctaccctgttgttgtagttctagctctatctccttttctaatcccccattcccatacttggacacaggactatagccaggaatggaaacagaattggcctgagaacaaccagagggtggtcgtgggggagggctggtgttcctggtgccttatccaccctcctcacccaccacctcctcaccagtctccctgcacacctccaccaccacagaaggagcctagggtggagctgggggggcatgaggtgatgaggagccagaaggagcccgtcagcacttttcagtgcccaaaataacaaagcaaaaggaagcacgcaggggtgcaaaggggcaggcggggcgaggggctgtgcccctacacctgggaggggtggcggggggagtaaaaaggcaggaaagagagagcagaagaggatgttcagaaacaagccgcggagcccgggttgggctgtggtgagtatctaggtcaccagggagcctgcaggcctgaccacagggagacctgtgttctcagctctcctcttcctccgaccctcccaaagaggcgaggtgaccccaatacagcttgaggccctctgctcgaccacccccagccccaagatctggaggccagaatccccagcctagactcattgcttgaattctgccatgattcaaatctgatttagaacttagaagattttgcttagcagattgaaatggcagactggacagaccccagaagcaaacaagagtgagaggtggacggggtcttgagcataagttccctaaaggctagagaagctgtgcttggagtcagccattcagagagcagcaagttaatcctttaatgaccaaatgcctcctgaccctgccctgtgccatgttctcctgcctcataaacccctggtccctggacctctttcagctgtcaagttcggccgcatgtccaagaagcagagggacagcctgcatgcagaagtgcagaaacagctgcagcagcggcaacagcagcaacaggaaccagtggtcaagacccctccagcaggggcccaaggagcagataccctcacctacaccttggggctcccagacgggcagctgcccctgggctcctcgcctgacctgcctgaggcttctgcctgtccccctggcctcctgaaagcctcaggctctgggccctcatattccaacaacttggccaaggcagggctcaatggggcctcatgccaccttgaatacagccctgagcggggcaaggctgagggcagagagagcttctatagcacaggcagccagctgacccctgaccgatgtggacttcgttttgaggaacacaggcatcctgggcttggggaactgggacagggcccagacagctacggcagccccagtttccgcagcacaccggaggcaccctatgcctccctgacagagataggtgagcagctggggaggtggagagggtggtagagatgagggaggggtttccaccagtaccccgtatcaatcaaacatgcgcctgagggaattgaggggtccagacgaggggcggagggaggaggcggagcaggataggccaggctgagaagtgcccttgcatgggtaggcgtaggagctggctgagatcaagccatgccttccttctccggccccagagcacctggtgcagagcgtctgcaagtcctacagggagacatgccagctgcggctggaggacctgctgcggcagcgctccaacatcttctcccgggaggaagtgactggctaccagaggaaggtgaggccaggagacctgcaggaagggaacgtatcccacccccaccgggagagttcagagatggctacctgcgcacgactgggtcctggggcagggcgggacatcacagacacaggctggccaacaagcatgtgcacaccttttatgtacagttgcgaatgtgtgtatctgtctacaccccagcagatggatgagcgcttttccttagttcttgcctattaaaattgtacctggccttcatcctacctcctctaccatgcagaccttggtgcaccctccttcctcctcttccatcaacaataacaataatcagaaccctgattaccatttgttaaacaccccttctctgccaggcattgtgctaaacgctttatgtaaattatcctcagcccctaccacaaccaacctatgagggtggcatttattcctacttttaacagacagaaactgagcctagaggggttaataggtttcctcaaagtcacaaagtggtggaaccaaagtcaaattcagattcatcaggctccaaagtttatgctgccttttcgatcacactctcataccacctgctctaaacacactcgtttggcacttcacatttgcttctccaggttattgagaccttgggcataacctttgtgggggaaggggcagctgtatgtcctgttcacctcaacgagatataccttgttggagggcaaagctgtatctcatactttttggattccctcacaacagctaactcggtgtcagcagccggtaggtgctcagtgtgtgggactcactggcaggaatctgtgcatttgtgctaagaccaggcttttgaaaatgctagttgagaacataggagttcagagcctaccccttgcagtttattaggtggggctccagggctcaggaggatcacagggccacacagagcgctacagcgggaccctcctccctccctgcagtccatgtgggagatgtgggaacggtgtgcccaccacctcaccgaggccattcagtacgtggtggagttcgccaagaggctctcaggctttatggagctctgccagaatgaccagattgtgcttctcaaagcaggtgcccagggatgggtgggcaggcctggggacaaggggacagagccaagtggagggaggtggcttaaggaaatcagggggacagagtcagatcctggctttgcttgacactgtccctgcatcttctctccccactgcccaggagcaatggaagtggtgctggttaggatgtgccgggcctacaatgctgacaaccgcacggtcttttttgaaggcaaatacggtggcatggagctgttccgagccttgggtgaggggcagggagaaatgagagggaagattctgatgccaaccccaggcaaagctttgtgacccagggcaccctcttttcagggcgaattgccccctctgctctaaacacaataagggcggtgtcctcgggcaccatcgctccagccactctctcacttttctcatttccactccatcaggctgcagcgagctcatcagctccatctttgacttctcccactccctaagtgccttgcacttttccgaggatgagattgccctctacacagcccttgttctcatcaatgcccgtgagtgttgctgggcttgggtgaaggacattcaggtggcaggggcatggcagatattgaagaagagtctagaccttcagatgtagttaaatctgggaaattgctttaaatagcagaatgagccctactcagtattgctataaaataaaatgagttaaaataaagattcagaggactctcagagagggacaagagcagcataaggtggggttgtgggaagtggggagaaatgaggttgagaggaaatgagccactttcctgacagaaatgtgtctgattgttagtctatggcagtgatttcattgtagcacacatcagaatcacctggggagctttaaaaactattgctgcctgggtcccacttccagagattccagtgtacatgtgctccatgacttatgatggggttatgtcccaataaacctatcgtaagttgaaaatatggtaagttgaaattgcatttaacacgcctaacttactgaacaccatagcttagcctagcctaccttaaatgtgctcagaatgcttacattagcctacagtctggcaaaagcatataacacaaagcctattttataataaagtgttgaatagctcatgtaatttattgaatatggttctaaaagtgaacagcaggatggttgcatgggtattcaaagtatggtttctactgaatgcaagtggctttctcaccaacataaaatcaaaaaatagtaagtcaactcatcataagactgggaccatctgtaattgataaggggtgcactgaattttaacagccacccccaggccaccaagattaagaatcactgctctgtgtgaactaattttaaggctgtatgcctataataggaagactctggatatcctatccactcccctggcatggagtagctgggctgagccagatgaatactaaatattcagagaacctagggaagtgggtcaagctgctaacctgagtttgcagatcgagactatcagtcttctggctctgccagtgagtggacacctaaatatgcccttcaaactagaagataaaaactttaaagatgacttctggatattttatagaatcagggcaaaagttagtcacagattgctgcaagccccctggtgcaggcctgggcaccttcaggaaggccacctcctatcaggagcccttttcgtacatgggggagctcttttccaactcttgttacccctatgacagaatcctagtgcctagcattgaatgagctatgtggaatatcaggttaccagggagaaggtagcttggagtcaggagatcagaaattctcatacctttcatctcttcccagttttccttgtagacagctagccaagcccaacgtccagagcttaccattatcaaaccaaggcacacacatgcacgcgctcagcttagaagacctctatccagcacagatgtccacaaagatacacccttttgttgggagttaatgtccatgttctttcttgttctcattacggtcccaccccctcctccagatcggccagggctccaagagaaaaggaaagtagaacagctgcagtacaatctggagctggcctttcatcatcatctctgcaagactcatcgccaaagcatcctggcaaaggtaggagcagtccctggggtagaagaggccaggcccatcgctagctctgtaacatcagagtttgcgagggccggggtctgtgggtacagaggagggagtgcgggagtaccactctctgttagagagcttgcatcagcagtgggaactaagggaatgaacagctacttccacgtgcataaagactggaaagttagagggcctgggattgggagggacctccagggaacaattcagtttaatatagccagcacttacccagcacctgcttgtacaaggcactgtggaaagacacagagatttggtcgctgcccccaccaagagattttaatctggtatgaagaagagatctgtgtatcactaactctaacatagagtagaatgtggtatgtgatataataataatgcaattaacagagtgcttttgctgacatgctttctcatcctcatggcaacccagtcaataggacaggtgttcaaatctccctgtgtagcagccgggcacagtggctcacgcctgtaatcccaacactttgggaggccaaggcgggtggatcacctgaggtcaggagttcgagaccagcctgactaacatggtgaaaccccatctctactaaaaataaaaaaaattagccaggcatggtggcaggcgcctgtagacccagctacttgggaggctgaggcaggataatcgcttgaaaccgggaggcagatgttgcagtgagccaagattgtgccattgcactccagcctgggcaacaagagcaaactctgtctcaaaaaaaaaaaaaaaatctccttgtagctatcaggagacttcagtgacttaaatgcaagattgaatcccagtgctctttgcgctctttctatccctgtgtcccctatgtataactataataagtgacaccaggaaaatgttatgagagtataaaacagggattaaaaataatttgggggtaaaaggagtgggtcataaatacttcccagggaagatgacatttatactaggccatgaatgatgtaagattttaacaggcattcatgggggtggggcaggcattccaggcttagggaacaataggagcaaaacaaaaaaaatgaaaaaaaatccttttcctgaggtttaaccaaaaaaatggatgagatgagtatgagaggctggggataattgttttatgggatttgggtgtgggactagggtacaatgaagaccaagaacaacaggagaaaaataaggaggcaaaatagtgtgtatgtggagaatcactcatggtacatcctcactaaagtgtaaaatcaggagctgggatagactggtggggcagaagagcaccagatgatcagcctgaaaattaggtcaggggcaaatcagagaggactttcaatgccatataagcgttaaagctttatttctaggccacagggagctccaaaggctagaaaaatgacacaatcagagctgtcatttaggcaatttactttggaaccagtataaaggaaccatttatgtaattattcaagcaggcctttgctatgtgccaggcacagggctgggtgttaaggatacagcaatgacttacacggtctgtgctctcaagaacttgaactttaatctgctacaggatggatttgaaggaggagagacaggaatctgggagagcaattggaaagcacatccggttggatctgctttcaaaatacatcaccttccctactattaccaccaccctggcccctaccagctctcagctttcacttggactttaagagaggcctcctaactgagcccctgttccaccctcatctgccctgtagcaaccacacctactccgcacccacggtagcccttttaaaaatgcaatctcatcatgccctactcctgtggtttttttcttgtttttgttgttgttgtttgtttgttctgtttgtttgttttgaggcagacttgctctgtcgcccaggctggagtgcagtggtgtgatctcggctcactgcaacctccacctcccaggttcaagcgattgtcctgcctcagcctcctgagtagcttggattacagggattacacgcccggctaatttttgtatttttagtagagacagggtttcaccatgttggccaggctggtctcgaactcctgacctcaggtgatctgcccaccttggcctcccaaagtgttgggattacaggcgtgaaccaccacacctggccttgactcctgttctcagccctcctgtagctgcctgtgatgccgagaatcaaatctagagtctgcgtcatggtcaagtggctcataacatgatccctgccttcttttctcacttgatcttccactgccccttcaaacacccattgcagccacacttgcttccttgctattcctcgaacacatcaaacccagtcgcagggcttttgtacctgctattgtagtcacctggagggttcttcccccagttttccaaatggcttaccccatctcttcattcgggagaggtttttcctgaccagtaaccccatacaaaaagctttagttttctttaaagaacttattatctgatacactacatatttattttctgggcccctcaccagaatgtaaatttaatcaaggtacagattatactttattgactgatatatctgatatatcactagaggccatcacagtgcctagctcagatccagatgtgttctcaacaaatatttgttgaatgaatgaaggaagctattgccatagcccaaaaaagctcagaataaagcagtggtgaggaagagagagaatctaggagatatgaagggatcacaccctgctgcctgcttttctctgactgcctctttccaaggaaactaagctgggggagggaggcaatggcaggcaagagacatttttaagcctcttggttgtagaggagactgcaagagataaattgttctttcagccttgctccagtgaggtctccctgcctccgtctgctcactggtttctgtgcctttttcatctcccctttggctgagtgctggcagcattggtttgctacttgcagtgttaggtgcctgcttagaaagtctgtttagttcaaagagttattaagcatgtgccatgtgctaagcattatgataggtacagaggagacagggaaagagagacctcagccaaggagctgaaatctagggtgggaaggcagacaaattggaccattttcctgcaatgtagtaagtgctacacagaggaaagcccaaaagaagggcccttaatccagatgggaggcagttagggaaatagtcttaggagaggtgacactagagggtaaggattagtaatgggaagaggggaacaacatgcacaaaggcagaggcatgtgacagcatacaacaagagtcacagacaatcactcttataaaagcaggaagtgcacattagagtctttgacaaggttcattctagaagtattgggaacataaattgagggcttcacccagaaaacattcacctgtgccccacccactctcacttccctccagtgtcctgaacacacacgtacttctaccagtgggatttggctggtgccaaagtgccaagtacataaggggaaggcaaggagggtttgtcctagcccaggaagaatgagcggacttctttgctctgaggagagctgaagtattgaccctcccttccccattaacccatatccagctgccacccaaggggaagcttcggagcctgtgtagccagcatgtggaaaggctgcagatcttccagcacctccaccccatcgtggtccaagccgctttccctccactctacaaggagctcttcagcactgaaaccgagtcacctgtggggctgtccaagtgacctggaagagggactccttgcctctccctatggcctgctggcccacctccctggaccccgttccaccctcacccttttcctttcccatgaaccctggagggtggtccccaccagctctttggaagtgagcagatgctgcggctggctttctgtcagcaggccggcctggcagtgggacaatcgccagagggtggggctggcagaacaccatctccagcctcagctttgacctgtctcatttcccatattccttcacacccagcttctggaaggcatggggtggctgggatttaaggacttctgggggaccaagacatcctcaagaaaacaggggcatccagggctccctggatgaatagaatgcaattcattcagaagctcagaagctaagaataagcctttgaaatacctcattgcatttccctttgggcttcggcttggggagatggatcaagctcagagactggcagtgagagcccagaaggacctgtataaaatgaatctggagctttacattttctgcctctgccttcctcccagctcagcaaggaagtatttgggcaccctaccctttacctggggtctaaccaaaaatggatgggatgaggatgagaggctggagataattgttttatgggatttgggtgtgggactagggtacaatgaaggccaagagcatctcagacatagagttaaaactcaaacctcttatgtgcactttaaagatagactttaggggctggcacaaatctgatcagagacacatatccatacacaggtgaaacacatacagactcaacagcaatcatgcagttccagagacacatgaacctgacacaatctctcttatccttgaggccacagcttggaggagcctagaggcctcaggggaaagtcccaatcctgagggaccctcccaaacatttccatggtgctccagtccactgatcttgggtctggggtgatccaaataccaccccagctccagctgtcttctaccactagaagacccaagagaagcagaagtcgctcgcactggtcagtcggaaggcaagatcagatcctggaggactttcctggcctgcccgccagccctgctcttgttgtggagaaggaagcagatgtgatcacatcaccccgtcattgggcaccgctgactccagcatggaggacaccagggagcagggcctgggcctgtttccccagctgtgatcttgcccagaacctctcttggcttcataaacagctgtgaaccctcccctgagggattaacagcaatgatgggcagtcgtggagttgggggggttgggggtgggattgtgtcctctaaggggacgggttcatctgagtaaacataaaccccaacttgtgccattctttataaaatgattttaaaggcaa

SEQ ID NO: 7 - DNA, 12152 bp, Artificial Sequence
  Artificially synthesized
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggactaggatcctttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgaggtaggcgtgtacggtgggaggcctatataagcagagctcgtttagtgaaccgtcagatcgcctggagacgccatccacgctgttttgacctccatagaagacaccgggaccgatccagcctccgcggccccgaactagtccagtgtggtggaattctgcagatatcaacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaagatctggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggttgatatccagcacagtggcggccgctcgagtctagagggcccgcggttcgaatacccatacgacgtcccagactacgcttagtaatgattaattaaactagaaattctaccgggtaggggaggcgcttttcccaaggcagtctggagtaacccacccaagatctggcctccgcgccgggttttggcgcctcccgcgggcgcccccctcctcacggcgagccgcgtaagatctggcctccgcgccgggttttggcgcctcccgcgggcgcccccctcctcacggcgagcctagtcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagcggccgccatgctagtaaattgtccgctaaattctggccgtttttggcttttttgttagacggatccataacttcgtataatgtatgctatacgaagttatgccaccatgtctagactggacaagagcaaagtcataaacggagctctggaattactcaatggtgtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaactaagtaaggatccgcggccgcactagaggaattccgcccctctcccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttaccggtccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgatgtacaagtaaagcggccgcgactctagatcataatcagccataccacatttgtagaggttttacttgctttaaaaaacctcccacacctccccctgaacctgaaacataaaatgaatgcaattgttgttgtttagtccctcccaattcgatatcaagcttatcgcgatagatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgagatcctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggcccgggttaattaaggaaagggctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcaagctcatggctgactaattttttttatttatgcagaggccgaggccgcctcggcctctgagctattccagaagtagtgaggaggcttttttggaggcctaggcttttgcaaaaagctccccgtggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 8 - DNA, 1878 bp, Homo Sapiens
aagcttgcaaatatatatatatatatatattttttttttttttttagagataagagtcttgctctgtcgcctaggctggagtgcagtggcacaatctctgctcactgcaacctccgcctccagggttcaagtgattctgctgcctcagcctcccaggtgggattacaggtgcctgccaccacgcctggctaatttttttgtctttttagtaaagatgaggtttcaccatgttgggcaggctggtttcaattgctgacctcaagtgagccaccccgcctcagcctcccaaaatgctaggattacaggcatgagccaccgcacccagccaagtttgtacatatatttttgactacacttcttaactattcttaggataaattactagaagtgaaaattctggggtgaagagcttgaggcctttacacacacacacacacacacacacacacacacaaataggctggatgcagtggctcacacctgtaatctcagcagtttgggaggctgaggaaggaggatcacttgagtccaggaggttgagaatagcctgaacaacatagcaagatcttgtctctacaaaaaattaaaaaaaaattagctggccatggcagcatgtgcctgtagtaccagctactcggaaggctgaggtaggaggatcgcttgagcccaggaggttgattgaagctgcagtgagctgtgattacaccactgcactccagcctgggcaacagagctagactctgtctctaaaaaaagcacaaaataatatttaaaaagcaccaggtatgcctgtacttgagttgtctttgttgatggctacaaatgaggacagctctggctgaagggcgcttccatttccatgggctgaaggagggacattttgcaaagtgtgttttcaggaagacacagagttttacctcctacacttgtttgatctgtattaatgtttgcttatttatttatttaattttttttttgagacagagtctcactctgtcacctgggctggagtgcagtggcattattgaggctcattgcagtctcagactcctgagctcaaacaatcctcctgcctcagcctctggagtagctaggactacaggcatgtgccaccatgcctggctaattttttaaatgtatttttttgtagagtcggggtctccctatgttgcccaggctggagtgcagtggtgtgatcctagctcactgcagcctggacctcgggctcaagtaattctcacacctcagcctgtccagtagcaggggctacaggcgcgcaccaccatgcccagctaattaaaaatatttttttgtagagacagggtctctctatgttgcccaggctggtttcaaactcccaggctcaagcaatcctcctgccttggcctcccaaagtgctggcattacaggcgtgagccactgcgcctggcccgtattaatgtttagaacacgaattccaggaggcaggctaagtctattcagcttgttcatatgcttgggccaacccaagaaacaagtgggtgacaaatggcaccttttggatagtggtattgactttgaaagtttgggtcaggaagctggggaggaagggtgggcaggctgtgggcagtcctgggcggaagaccaggcagggctatgtgctcactgagcctccgccctcttcctttgaatctctgatagacttctgcctcctacttctccttttctgcccttctttgctttggtggcttccttgtggttcctcagtggtgcctgcaacccctggttcacctccttccaggttctggctccttccagccatggctctcagagtccttctgttaacaggtgcatgggggtggggtgggggactctgggtggggaggagggtaacttttg

SEQ ID NO: 9 - DNA, 1557 bp, Homo Sapiens
atggacagggccccacagagacagcaccgagcctcacgggagctgctggctgcaaagaagacccacacctcacaaattgaagtgatcccttgcaaaatctgtggggacaagtcgtctgggatccactacggggttatcacctgtgaggggtgcaagggcttcttccgccggagccagcgctgtaacgcggcctactcctgcacccgtcagcagaactgccccatcgaccgcaccagccgaaaccgatgccagcactgccgcctgcagaaatgcctggcgctgggcatgtcccgagatgctgtcaagttcggccgcatgtccaagaagcagagggacagcctgcatgcagaagtgcagaaacagctgcagcagcggcaacagcagcaacaggaaccagtggtcaagacccctccagcaggggcccaaggagcagataccctcacctacaccttggggctcccagacgggcagctgcccctgggctcctcgcctgacctgcctgaggcttctgcctgtccccctggcctcctgaaagcctcaggctctgggccctcatattccaacaacttggccaaggcagggctcaatggggcctcatgccaccttgaatacagccctgagcggggcaaggctgagggcagagagagcttctatagcacaggcagccagctgacccctgaccgatgtggacttcgttttgaggaacacaggcatcctgggcttggggaactgggacagggcccagacagctacggcagccccagtttccgcagcacaccggaggcaccctatgcctccctgacagagatagagcacctggtgcagagcgtctgcaagtcctacagggagacatgccagctgcggctggaggacctgctgcggcagcgctccaacatcttctcccgggaggaagtgactggctaccagaggaagtccatgtgggagatgtgggaacggtgtgcccaccacctcaccgaggccattcagtacgtggtggagttcgccaagaggctctcaggctttatggagctctgccagaatgaccagattgtgcttctcaaagcaggagcaatggaagtggtgctggttaggatgtgccgggcctacaatgctgacaaccgcacggtcttttttgaaggcaaatacggtggcatggagctgttccgagccttgggctgcagcgagctcatcagctccatctttgacttctcccactccctaagtgccttgcacttttccgaggatgagattgccctctacacagcccttgttctcatcaatgcccatcggccagggctccaagagaaaaggaaagtagaacagctgcagtacaatctggagctggcctttcatcatcatctctgcaagactcatcgccaaagcatcctggcaaagctgccacccaaggggaagcttcggagcctgtgtagccagcatgtggaaaggctgcagatcttccagcacctccaccccatcgtggtccaagccgctttccctccactctacaaggagctcttcagcactgaaaccgagtcacctgtggggctgtccaagtga

(19) United States
(12) Patent Application Publication
(10) Pub. No.: US 2017/0218036 A1
(43) Pub. Date: Aug. 3, 2017

(54) INNATE IMMUNE SYSTEM MODIFICATION FOR ANTICANCER THERAPY

(71) Applicant: YALE UNIVERSITY, New Haven, CT (US)

(72) Inventors: Elias THEODOROU, Hamden, CT (US); Michael SNYDER, Stanford, CA (US); Joseph A. MADRI, North Branford, CT (US)

(21) Appl. No.: 15/515,075

(22) PCT Filed: Sep. 29, 2015
(86) PCT No.: PCT/US15/52898

§ 371 Date: Mar. 28, 2017

Related U.S. Application Data
(60) Provisional application No. 62/059,342, filed on Oct. 3, 2014.

(51) Int. Cl. C07K 14/47; C12Q 1/68; A61K 39/00

(52) U.S. Cl. CPC C07K 14/4702; A61K 39/0011; C12Q 1/6886; A61K 2039/5156; C12Q 2600/158; A61K 2039/572

(57) ABSTRACT

The present invention relates to the discovery of a role of the nuclear receptor retinoid-related orphan receptor gamma (RORgamma) in tumor suppression. The introduction and expression of RORgamma result in genes activation within innate immune cells that trigger recognition and suppression of tumor cells. Thus, in various embodiments described herein, the invention encompasses a composition or a cell comprising a viral vector comprising nucleic acid sequences encoding RORgamma under the control of a neutrophil specific promoter. Additionally, the invention relates to methods of treating cancer by administering to a subject a composition that confers or increases innate immune cell anti-tumor immunity, methods for providing anti-tumor immunity in a subject, methods of stimulating innate immune response to a cell population or a tissue in a mammal and methods of diagnosing anti-tumor immunity response. Furthermore, the invention encompasses a kit for carrying out the aforementioned methods.

### CROSS-REFERENCE TO RELATED APPLICATIONS

The present application is a 35 U.S.C. §371 national phase application from, and claims priority to, International Application No. PCT/US2015/052898, filed Sep. 29, 2015, and published under PCT Article 21(2) in English, which claims priority under 35 U.S.C. §119(e) to U.S. Provisional Patent Application No. 62/059,342, filed Oct. 3, 2014, all of which applications are incorporated herein by reference in their entireties.

### BACKGROUND OF THE INVENTION

The past few decades have seen remarkable advancements in cancer treatment and diagnosis. Current treatment options for cancer includes surgery, chemotherapy, radiation therapy, and immunotherapy. Most recently, immunotherapy treatment, aiming at stimulating the immune system, has registered promising successes in the fight against cancer and has attracted numerous investigations.

Although immunotherapy can be highly efficacious, only small subsets of patients, regardless of the organ of origin of the tumor, are usually responsive to therapy. Furthermore, some of these treatments are highly personalized and impossible to administer outside of specialized settings which makes them extraordinarily expensive. New findings in this field are clearly needed for improving immunotherapy efficacy to any given type of cancer and for making it more accessible and more affordable.

It has been reported that cells of the innate immune system (both mouse and human) have an inherent ability to both target and kill cancer cells of various lineages. Though this phenomenon had been recognized for years, the mechanism(s) guiding this cancer killing ability have eluded researchers (Cui et al., Proc Natl Acad Sci USA. 2003; 100(11):6682-7; Hicks et al., Proc Natl Acad Sci USA. 2006; 103(20):7753-8; Sanders et al., -BMC Cancer. 2010; 10:121).

More than a decade ago, mice resistant to cancer, designated as SR/CR, were discovered to be immune to tumor development when challenged with increasing doses of cancerous sarcoma 180 (S180) cells that would be devastating to normal mice. The cancer resistance trait proved to be heritable and from a single mouse generations of SR/CR mice were bred (Cui et al., Proc Natl Acad Sci USA. 2003; 100(11):6682-7; Hicks et al., Proc Natl Acad Sci USA. 2006). Unfortunately the key genes responsible for the phenotype were not readily identified (Riedlinger et al., BMC Cancer. 2010; 10:179).

At the cellular level, the surprising finding was that the anticancer activity of SR/CR mice was dominated by the innate immune system (e.g. neutrophils, natural killer cells and macrophages). Neutrophils and macrophages, the first responders of the immune system, were responsible for moving toward and killing cancer cells. This was a surprising find since almost all of the literature related to anticancer immunity had been based on the adaptive immune system (e.g. T cells) and not the innate immune system. The most astounding set of experiments involved the transfer of SR/CR white blood cells to mice with prostate specific knockout of PTEN tumor suppressor gene. PTEN knockout mice normally succumb to prostate tumors 100% of the time. PTEN mutant mice treated with white blood cells from SR/CR mice outlived all the control, untreated PTEN mice (Sanders A. M., 2007, Thesis: “Characterization of the Inheritance, Effector Mechanisms, and Response Against Endogenous Cancers in the SR/CR mouse Model of Cancer Resistance”; Wake Forest University). Experiments in human neutrophils and macrophages with in vitro cancer killing assays showed varying degrees of efficiency in the attack of target cancer cell lines and that healthy young subjects had more efficient killing activity compared to older subjects. However, the key players in the white blood cells remained unidentified (Koch et al., APMIS. 2012; 120(12):974-87).

Clearly, there is a need for new modalities to arrest cancer cell proliferation, to trigger cancer cell death, and to therefore treat cancer. The current invention fulfills this need. Furthermore, the present invention satisfies the need for a universal, broad anti-tumor immunotherapy, regardless of the type of cancer.

### SUMMARY OF THE INVENTION

As described below, the present invention features compositions and methods of modifying an innate immune system for anticancer therapy.

In one aspect, the invention provides a composition comprising a viral vector comprising a nucleic acid sequence encoding retinoid-related orphan receptor gamma (RORgamma), wherein expression of RORgamma is under the control of a neutrophil specific promoter.

In another aspect, the invention provides a cell comprising a viral vector comprising a nucleic acid sequence encoding RORgamma, wherein expression of RORgamma is under the control of a neutrophil specific promoter.

In yet another aspect, the invention provides a CD34 positive (CD34⁺) cell comprising a viral vector comprising a nucleic acid sequence encoding RORgamma, wherein expression of RORgamma is under the control of a promoter selected from the group consisting of a neutrophil specific promoter, a constitutive promoter, and an inducible promoter.

In still another aspect, the invention provides a CD34⁺ cell that is committed to differentiate into a neutrophil, the cell comprising a viral vector comprising a nucleic acid sequence encoding RORgamma, wherein expression of RORgamma is under the control of a promoter selected from the group consisting of a neutrophil specific promoter, a constitutive promoter, and an inducible promoter.

In another aspect, the invention provides a method of treating a cancer in a mammal in need thereof, the method comprising administering to the mammal a composition comprising a viral vector comprising a nucleic acid sequence encoding retinoid-related orphan receptor gamma (RORgamma), wherein expression of RORgamma is under the control of a promoter selected from the group consisting of a neutrophil specific promoter, a constitutive promoter, and an inducible promoter.

In yet another aspect, the invention provides a method of treating a cancer in a mammal in need thereof, the method comprising administering to the mammal a cell comprising a viral vector comprising a nucleic acid sequence encoding RORgamma, wherein expression of RORgamma is under the control of a promoter selected from the group consisting of a neutrophil specific promoter, a constitutive promoter, and an inducible promoter.

In still another aspect, the invention provides a method of treating a cancer in a mammal in need thereof, the method comprising administering to the mammal a CD34 positive (CD34⁺) cell comprising a viral vector comprising a nucleic acid sequence encoding RORgamma, wherein expression of ROR gamma is under the control of a promoter selected from the group consisting of a neutrophil specific promoter, a constitutive promoter, and an inducible promoter.

In another aspect, the invention provides a method of treating a cancer in a mammal in need thereof, the method comprising administering to the mammal a CD34⁺ cell that is committed to differentiate into a neutrophil, the cell comprising a viral vector comprising a nucleic acid sequence encoding RORgamma, wherein expression of RORgamma is under the control of a promoter selected from the group consisting of a neutrophil specific promoter, a constitutive promoter, and an inducible promoter.

In still another aspect, the invention provides a method for providing anti-tumor immunity in a mammal, the method comprising administering to the mammal a composition comprising a viral vector comprising a nucleic acid sequence encoding RORgamma, wherein expression of RORgamma is under the control of a promoter selected from the group consisting of a neutrophil specific promoter, a constitutive promoter, and an inducible promoter.

In yet another aspect, the invention provides a method for stimulating innate immune response to a cell population or tissue in a mammal, the method comprising administering to a mammal a composition comprising a viral vector comprising a nucleic acid sequence encoding RORgamma, wherein expression of RORgamma is under the control of a promoter selected from the group consisting of a neutrophil specific promoter, a constitutive promoter, and an inducible promoter.

In various embodiments of any of the aspects delineated herein, the viral vector is selected from the group consisting of a lentiviral vector, retroviral vector, adenoviral vector, and adeno-associated viral (AAV) vector. In various embodiments, the neutrophil specific promoter is CD11B (SEQ ID NO. 8).

In another aspect, the invention provides a method of diagnosing the activation or activity of anti-tumor immunity in a mammal in need thereof, the method comprising determining the expression level of retinoid-related orphan receptor gamma (RORgamma) in a biological sample from the mammal, wherein an increase in the expression level of RORgamma as compared with a normal control level of RORgamma expression is an indication that the mammal has or is developing anti-tumor immunity.

In various embodiments of any of the aspects delineated herein, the biological sample comprises at least one selected from the group consisting of blood, white blood cells and neutrophils. In various embodiments, the expression level is at least 10% greater than the normal control level. In various embodiments, the expression level is determined by a method selected from the group consisting of detecting mRNA of the gene, detecting a protein encoded by the gene, and detecting a biological activity of the protein encoded by the gene.

In various embodiments of any of the aspects delineated herein, the mammal is a human.

In still another aspect, the invention provides a kit comprising probe sets for the retinoid-related orphan receptor gamma (RORgamma) and instructions for use thereof, wherein the instructions comprise detecting the level of RORgamma in innate immune cells in a sample from a mammal in need thereof; providing an indication on presence or absence of anti-tumor immunity; and providing a recommendation of whether or not anti-tumor immunity treatment comprising administering to the mammal a composition comprising a viral a nucleic acid sequence encoding RORgamma, wherein expression of RORgamma is under the control of a promoter selected from the group consisting of a neutrophil specific promoter, a constitutive promoter, and an inducible promoter, should be administered, modified, or terminated in the mammal.

Compositions and articles defined by the invention were isolated or otherwise manufactured in connection with the examples provided below. Other features and advantages of the invention will be apparent from the detailed description, and from the claims.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 represents a summary of the work flow that included RNA-Seq of SR/CR and control samples and an in vitro functional assay. Thioglycollate-elicited peritoneal neutrophils were harvested from control and SR/CR mice (>85% pure based on morphological analysis of Giemsa stained samples prepared by Cytospin). Total RNA was harvested and used to perform RNA-Seq. Analysis of RNA-Seq data led to the functional testing of several candidate differentially upregulated at the RNA level in the SR/CR neutrophils and determination of RORgamma as an effector of the SR/CR cancer killing mechanism.

FIGS. 2A-2B are scatterplot graphs demonstrating the RNA-Seq correlation of replicates. Data demonstrates the high consistency of overlap between individual control/WT replicates (FIG. 2A) and SR/CR replicated (FIG. 2B). WT1 vs WT3, SR/CR1 vs SR/CR3, etc. were equally consistent.

FIG. 3 is a graph illustrating the principle component analysis (PCA) between wild (WT) and SR/CR mouse.

FIG. 4 is a table illustrating the top differential display positives from RNA-Seq data. Levels of coding and non-coding RNA that were deemed important had a false discovery rate (FDR) score of <0.05. Of note are RORgamma (Rorc) and class B scavenger receptors (Scart2, WC1.1, Cd6) as well as CD148 tyrosine phosphatase receptor. RORgamma was the only transcription factor that met the criteria of a) >2 fold increase in expression and b) FDR value of <0.5 (initial P-value for RORgamma was 0.00065).

FIG. 5 is a plot demonstrating that the RORgamma is a marker of the SR/CR trait. At the RNA level, RORgamma was consistently higher in neutrophils from SR/CR mice vs neutrophils from control mice. This was obtained in neutrophils isolated from C57BL/6 mice, an inbred strain of mice that the original SR/CR BALB/c mice were backcrossed onto.

FIG. 6 is a series of images illustrating lentiviral expression of a control transgene in HF1 myeloid precursors cells. Green fluorescent protein (GFP) was delivered via the Mach7 lentivirus. It was found that the expression of Mach7-GFP, which was driven by the constitutive promoter, hEFlalpha, was nearly ubiquitous in the target HF1 cell line. The assay confirmed that levels of transgene expression by an in-house lentiviral vector in the HF1 cell line model was adequate for further assay development. Visualization of GFP via fluorescence microscopy confirmed robust expression of transgenes delivered via lentivirus in HF1 cells.

FIG. 7 is a series of images demonstrating that HF1 cells efficiently differentiate into neutrophils. Using a one-step protocol the HF1 neutrophils (whether expressing a transgene or not), efficiently differentiated into neutrophils as determined using the anti-Ly-6G, Ly-6C rat antibody (NIMP-R14, Abcam).

FIG. 8 is a schematic representing an overview of the functional cell biology assay for ability of proteins and/or RNAs to confer cancer cell killing on neutrophils. Lentivirus encoding RORgamma is used to infect HF1 progenitor cells. Antibiotic is added to kill off any HF1 progenitors that are not infected with lentivirus. HF1-RORgamma transgenic myeloid progenitor cells are then differentiated with G-CSF, then added to a plate of a monolayer of Renca cancer cells. The HF1-RORgamma neutrophils to do not adhere to the plastic dish but have access to Renca cells. After 48 hours, the neutrophils are washed away and the Renca cells are stained with Crystal Violet to compare with control cells for loss of viability.

FIG. 9 is an image demonstrating that neutrophils infected with Lentivirus expressing mouse RORgamma (mRORgamma) reduce the number of cancer cells in an in vitro assay. Renca cells were allowed to adhere and grow overnight to a tissue culture treated 6-well dish for 24 hr. Then, HF1-mRORgamma neutrophils were added at varying ratios of neutrophils:Renca cells—1:1, 10:1 and 20:1. After a 48 hr incubation period, media containing neutrophils was aspirated and plates were washed with PBS before simultaneously fixing and staining Renca cells with 1% Crystal Violet in 50% ethanol. HFI-mRORgamma cells consistently cleared more Renca cells than controls.

FIG. 10 is an overview of the pinducer21 inducible Lentivirus. mRORgamma was cloned downstream of the TRE2 inducible promoter. Cells infected with this construct produce mRORgamma upon the addition of doxycycline and the activation of the constitutively expressed rtTA3 transcriptional transactivation protein.

FIGS. 11A-11C are images demonstrating the ability to differentiate the HoxA9 HF1 cell line into neutrophils. HF1 cells were maintained in standard growth media or washed with saline (FIG. 11A) and grown in the presence of 20 ng/mL of G-CSF for three days (FIG. 11B) or for six days (FIG. 11C). Cells were cytospun at indicated times and stained with Giemsa. Arrows indicate cells that clearly demonstrate multilobed nuclei characteristic of neutrophils.

FIGS. 12A-12H are images demonstrating that immune cells modified with pInducer21 Lentivirus RORgamma are able to reduce the number of cancer cells in an in vitro assay. Renca cells were allowed to adhere and grow overnight to a tissue culture treated 6-well dish for 24 hours. Then, differentiated HF1-inducible RORgamma cells were added at a ratio of 20:1 (neutrophils:Renca) without (minus doxycycline (FIGS. 12A and 12E) or with (plus doxycycline FIGS. 12B, 12C, 12D, 12F, 12G, and 12H). After a 48 hr incubation period, media containing neutrophils was aspirated and plates were washed with PBS before fixing Renca cells with 4% paraformaldehyde with PBS. Transgenic HF1 cells that were both differentiated and induced for RORgamma expression via doxycycline consistently cleared more Renca cells than differentiated HF1 transgenic cells in which RORgamma was not induced with doxycycline.

FIG. 13 is a diagram showing a detailed overview of the Mach7 constitutive Lentiviral vector. The Mach7 Lentiviral vector drives expression of human RORgamma (hRORgamma) via the human EF1alpha promoter. In the antisense orientation relative to the hEFlalpha promoter, a human PGK promoter drives production of the blasticidin resistance protein. The majority of target cells infected with this construct and selected for with blasticidin express hRORgamma.

FIGS. 14A-14D are images demonstrating the differentiation and expression of human RORgamma in a human model of neutrophil differentiation. NB4 cells were infected with Mach7-hRORgamma Lentivirus and stained with anti-HA antibody to visualize hRORgamma (FIG. 14A, inset). NB4 cells that survived blasticidin selection was positive for hRORgamma. Cells were further differentiated with all trans retinoic acid (ATRA) and after six days cytospun onto slides and stained with Giemsa (FIGS. 14B-14D). Further, cells at Day 6 were subjected to a nitroblue tetrazolium assay (NBT) in order to test the ability of differentiated cells to reduce nitroblue tetrazolium, a hallmark of differentiated neutrophils. Cells were stimulated with phorbal myristate acetate (PMA) for 30 minutes and subsequently cytospun and fixed with 4% paraformaldehyde. Reduction of NBT resulted in a black precipitate (FIG. 14D, inset) in approximately 10% of cells, as reported in the literature for normal differentiation.

FIGS. 15A-15B are listings of an exemplary human RORgamma native amino acid sequence (FIG. 15A; SEQ ID NO: 1. GenBank: CAG33717.1; NCBI ref. NP 005051.2; Swiss-Prot ref. P51449.2) and an exemplary human RORgamma cDNA nucleic acid sequence (FIG. 15B; SEQ ID NO: 9. GenBank: CAG33717.1; NCBI ref. NM_005060.3).

FIG. 16 is a Mach7 lentiviral vector nucleic acid sequence (SEQ ID NO: 2).

FIG. 17 is a mouse RORC nucleic acid sequence (SEQ ID NO: 3).

FIG. 18 is a mouse RORgamma amino acid sequence (SEQ IDs NO: 4 and 5). SEQ ID NO: 4 corresponds to RORgamma long isoform that showed functional results in the killing assays. This isoform is mainly expressed in fat and muscle cells and was surprisingly found to be active in immune cells in the present invention. SEQ ID NO: 5 corresponds to RORgamma short isoform and was inactive in the killing assays.

FIG. 19 is a human RORC (hRORC) nucleic acid sequence (SEQ ID NO: 6). This sequence is an illustration of many possible variants of the hRORC.

FIG. 20 is a plasmid pinducer21 (also known as pICEE-DEST) nucleic acid sequence (SEQ ID NO: 7).

FIG. 21 is a promoter CD11B nucleic acid sequence (SEQ ID NO: 8).

### DETAILED DESCRIPTION OF THE INVENTION

The present invention relates to the unexpected discovery of a role of the nuclear receptor retinoid-related orphan receptor gamma (RORgamma) in tumor suppression. The introduction and expression of RORC gene, encoding RORgamma protein, results in gene activation within innate immune cells that triggers recognition and suppression of tumor cells. Thus, in various embodiments described herein, the invention encompasses a composition or a cell comprising a viral vector comprising nucleic acid sequences encoding RORgamma under the control of a neutrophil specific promoter. Additionally, the invention relates to methods of treating cancer by administering to a subject a composition that confers or increases innate immune cell anti-tumor immunity, methods for providing anti-tumor immunity in a subject, methods of stimulating innate immune response to a cell population or a tissue in a mammal and methods of diagnosing anti-tumor immunity response. Furthermore, the invention encompasses a kit for carrying out the aforementioned methods.

### Definitions

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

As used herein, the articles “a” and “an” are used to refer to one or to more than one (i.e., to at least one) of the grammatical object of the article. By way of example, “an element” means one element or more than one element.

As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

As used herein, “10% greater” refers to expression levels which are at least 10% or more, for example, 20%, 30%, 40%, or 50%, 60%, 70%, 80%, 90% higher or more, and/or 1.1 fold, 1.2 fold, 1.4 fold, 1.6 fold, 1.8 fold, 2.0 fold higher or more, and any and all whole or partial increments there between, than a control.

As used herein, the terms “control,” or “reference” are used interchangeably, and refer to a value that is used as a standard of comparison (e.g., RORC level of expression in a healthy subject).

A “subject” or “patient,” as used therein, may be a human or non-human mammal. Non-human mammals include, for example, livestock and pets, such as ovine, bovine, porcine, canine, feline and murine mammals. Preferably, the subject is human.

A “mutation” as used therein is a change in a DNA sequence resulting in an alteration from its natural state. The mutation can comprise deletion and/or insertion and/or duplication and/or substitution of at least one deoxyribonucleic acid base such as a purine (adenine and/or thymine) and/or a pyrimidine (guanine and/or cytosine) Mutations may or may not produce discernible changes in the observable characteristics (phenotype) of an organism (subject).

The term “immunogenicity” as used herein, is the ability of a particular substance, such as an antigen or epitope, to provoke an immune response in the body of a mammal. This immune response could be humoral and/or cell-mediated.

The term “activation”, as used herein, refers to the state of a cell following sufficient cell surface moiety ligation to induce a noticeable biochemical or morphological change. Within the context of immune cells, the activation is the transition of leucocytes and other immune cells. Within the context of T cells, such activation refers to the state of a T cell that has been sufficiently stimulated to induce cellular proliferation. Activation of a T cell may also induce cytokine production and performance of regulatory or cytolytic effector functions. Within the context of other cells, this term infers either up or down regulation of a particular physico-chemical process.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that may comprise a protein or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “RNA” as used herein is defined as ribonucleic acid.

The term the “immunotherapeutic agent” as used herein is meant to include any agent that modulates the patient's immune system. “immunotherapy” refers to the treatment that alters the patient's immune system.

The term “therapeutic” as used herein means a treatment and/or prophylaxis. A therapeutic effect is obtained by suppression, remission, or eradication of a disease state.

The term “treatment” as used within the context of the present invention is meant to include therapeutic treatment as well as prophylactic, or suppressive measures for the disease or disorder. Thus, for example, the term treatment includes the administration of an agent prior to or following the onset of a disease or disorder thereby preventing or removing all signs of the disease or disorder. As another example, administration of the agent after clinical manifestation of the disease to combat the symptoms of the disease comprises “treatment” of the disease. This includes prevention of cancer.

The term “biological sample” refers to a sample obtained from an organism or from components (e.g., cells) of an organism. The sample may be of any biological tissue or fluid. Frequently the sample will be a “clinical sample” which is a sample derived from a patient. Such samples include, but are not limited to, bone marrow, cardiac tissue, sputum, blood, lymphatic fluid, blood cells (e.g., white cells), tissue or fine needle biopsy samples, urine, peritoneal fluid, and pleural fluid, or cells therefrom. Biological samples may also include sections of tissues such as frozen sections taken for histological purposes.

“RORgamma” refers to a Retinoid (RAR) related orphan receptor gamma protein amino acid sequence encoded by RORC gene. RORgamma is member of the nuclear receptor family of transcription factors. These proteins function as key regulators of many physiological processes that occur during embryonic development and in the adult. In some instances, the term “RORgamma” and “RORC” are used interchangeably. The term “RORgamma” may be used to refer to the RORgamma gene nucleic acids sequence. RORgamma nucleic acid sequence may also be referred to herein as “RORC”.

By “mouse RORgamma protein” is meant a polypeptide having at least about 85% amino acid sequence identity to SEQ. ID. NO. 4 (Isoform 1) or SEQ. ID. NO. 5 (RORgamma-t or Isoform 2) or a fragment thereof having a biological function or activity of RORgamma. Examples of biological function or activity of RORgamma include, but are not limited to, transcription factor activity and regulation of physiological process that occur during embryonic development and in the adult.

By “mouse RORgamma polynucleotide” is meant a nucleic acid sequence encoding a mouse RORgamma protein. An exemplary mouse RORgamma polynucleotide sequence is provided at SEQ. ID. NO. 3.

By “human RORgamma protein” is meant a polypeptide having at least about 85% amino acid sequence identity to NCBI Ref. No. NP 005051.2 (SEQ. ID. NO. 1) or NCBI Ref. No. NP 001001523 or a fragment thereof having a biological function or activity of RORgamma. Examples of biological function or activity of RORgamma include, but are not limited to, transcription factor activity and regulation of physiological process that occur during embryonic development and in the adult.

By “human RORgamma polynucleotide” is meant a nucleic acid sequence encoding a mouse RORgamma protein. An exemplary human RORgamma polynucleotide sequence is provided at SEQ ID. NO. 9.

A “host,” as the term is used herein, includes prokaryotic or eukaryotic organisms that can be genetically engineered. For examples of such hosts, are found in Maniatis et al., Molecular Cloning. A Laboratory Manual, Cold Spring Harbor Laboratory, Cold Spring Harbor, N.Y. (2012). The terms “host,” “host cell,” “host system” and “expression host” are used interchangeably herein.

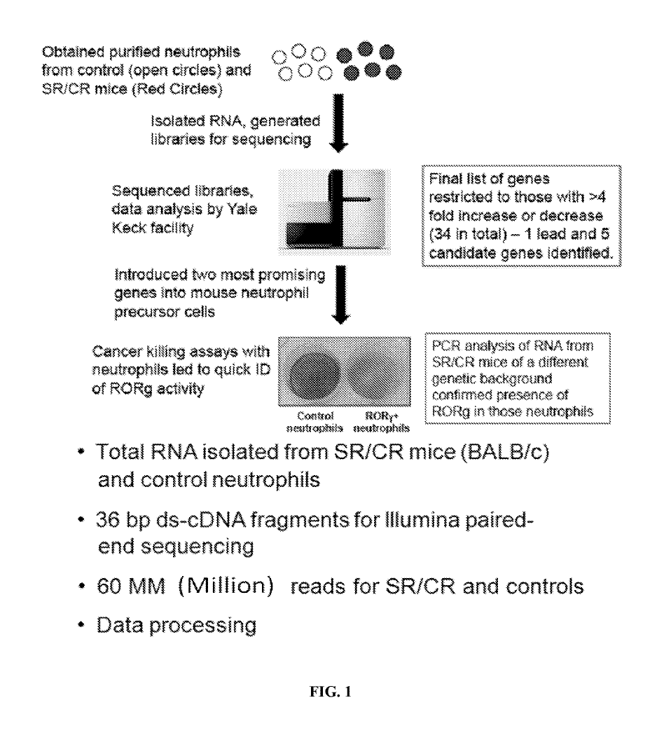

The term “recognition sequence” refers to particular DNA sequences which are recognized (and bound by) a protein, DNA, or RNA molecule, including a restriction endonuclease, a modification methylase, and a recombinase. For example, the recognition sequence for Cre recombinase is loxP which is a 34 base pair sequence comprised of two 13 base pair inverted repeats (serving as the recombinase binding sites) flanking an 8 base pair core sequence. See FIG. 1 of Sauer, B., Current Opinion in Biotechnology 5:521-527 (1994). Other examples of recognition sequences are the attB, attP, attL, and attR sequences which are recognized by the integrase of bacteriophage lambda. AttB is an approximately 25 base pair sequence containing two 9 base pair core-type Int binding sites and a 7 base pair overlap region. attP is an approximately 240 base pair sequence containing core-type Int binding sites and arm-type Int binding sites as well as sites for auxiliary proteins IHF, FIS, and Xis. See Landy, Current Opinion in Biotechnology 3:699-707 (1993). Such sites are also engineered according to the present invention to enhance methods and products.

The term “recombinase” refers to an enzyme which catalyzes the exchange of DNA segments at specific recombination sites.

As used herein, the term “exogenous” refers to any material introduced from or produced outside an organism, cell, tissue or system.

The term “expression” as used herein is defined as the transcription and/or translation of a particular nucleotide sequence driven by its promoter.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo and ex vivo.

The term “equivalent,” when used in reference to nucleotide sequences, is understood to refer to nucleotide sequences encoding functionally equivalent polypeptides. Equivalent nucleotide sequences will include sequences that differ by one or more nucleotide substitutions, additions- or deletions, such as allelic variants; and will, therefore, include sequences that differ from the nucleotide sequence of the nucleic acids described herein due to the degeneracy of the genetic code.

“Hybridization” refers to any process by which a strand of nucleic acid binds with a complementary strand through base pairing. Two single-stranded nucleic acids “hybridize” when they form a double-stranded duplex. The region of double-strandedness can include the full-length of one or both of the single-stranded nucleic acids, or all of one single stranded nucleic acid and a subsequence of the other single stranded nucleic acid, or the region of double-strandedness can include a subsequence of each nucleic acid. Hybridization also includes the formation of duplexes, which contain certain mismatches, provided that the two strands are still forming a double stranded helix. “Stringent hybridization conditions” refers to hybridization conditions resulting in essentially specific hybridization. The term “specific hybridization” of a probe to a target site of a template nucleic acid refers to hybridization of the probe predominantly to the target, such that the hybridization signal can be clearly interpreted. As further described herein, such conditions resulting in specific hybridization vary depending on the length of the region of homology, the GC content of the region, the melting temperature “Tm” of the hybrid. Hybridization conditions will thus vary in the salt content, acidity, and temperature of the hybridization solution and the washes.

The term “isolated” as used herein with respect to nucleic acids, such as DNA or RNA, refers to molecules separated from other DNAs or RNAs, respectively that are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments, which are not naturally occurring as fragments and would not be found in the natural state. The term “isolated” is also used herein to refer to polypeptides, which are isolated from other cellular proteins and is meant to encompass both purified and recombinant polypeptides. An “isolated cell” or “isolated population of cells” is a cell or population of cells that is not present in its natural environment.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids.

By “substantially identical” or “substantially homologous” is meant a polypeptide or nucleic acid molecule exhibiting at least 60% identity to a reference amino acid sequence (for example, any one of the amino acid sequences described herein) or nucleic acid sequence (for example, any one of the nucleic acid sequences described herein). Preferably, such a sequence is at least 70%, more preferably 80% or 85%, and more preferably 90%, 95% or even 99% identical at the amino acid level or nucleic acid to the sequence used for comparison. “Percent identity” or “percent homology” are used interchangeably herein.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence, which is required for expression of a gene product operably linked to the promoter/regulatory sequence. In some instances, this sequence may be the core promoter sequence and in other instances, this sequence may also include an enhancer sequence and other regulatory elements, which are required for expression of the gene product. The promoter/regulatory sequence may, for example, be one that expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a cell substantially only if the cell is a cell of the tissue type corresponding to the promoter.

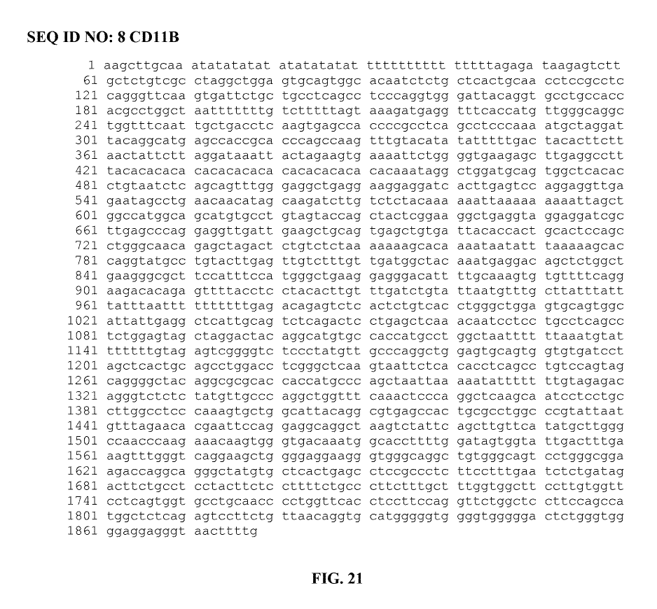

A “neutrophil specific promoter” is a nucleotide sequence which, when operably linked with a polynucleotide which encodes or specifies a gene product, is capable of driving expression of the gene product in a neutrophil or a cell that is committed to differentiate into a neutrophil. In some embodiments, expression of RORgamma is under the control of a neutrophil specific promoter. In some other embodiments, the neutrophil specific promoter causes the gene product to be produced in a cell substantially only if the cell is a neutrophil or cell that is committed to differentiate into a neutrophil. Examples of a neutrophil specific promoter include, but are not limited to, the integrin alpha promoter CD11B (See FIG. 21, SEQ ID NO: 8; Shelley and Arnaout, Proc Natl Acad Sci USA. 1991; 88(23):10525-9) or the alpha defensin promoters DEFA1, DEFA2, DEFA3, and DEFA4.

A “stem cell” refers to a cell that is capable of differentiating into a desired cell type. A stem cell includes embryonic stem (ES) cells; adult stem cells; and somatic stem cells, such as SP cells from uncommitted mesoderm. A “totipotent” stem cell is capable of differentiating into all tissue types, including cells of the meso-, endo-, and ecto-derm. A “multipotent” or “pluripotent” stem cell is a cell that is capable of differentiating into at least two of several fates.

The term “variant,” when used in the context of a polynucleotide sequence, may encompass a polynucleotide sequence related to that of a gene or the coding sequence thereof. This definition may also include, for example, “allelic,” “splice,” “species,” or “polymorphic” variants. The polypeptides generally will have significant amino acid identity relative to each other. A polymorphic variant is a variation in the polynucleotide sequence of a particular gene between individuals of a given species. Polymorphic variants may encompass “single nucleotide polymorphisms” (SNPs) in which the polynucleotide sequence varies by one base. The presence of SNPs may be indicative of, for example, a certain population, a disease state, or a propensity for a disease state.

The term “cancer” as used herein, includes any malignant tumor including, but not limited to, carcinoma, sarcoma. Cancer arises from the uncontrolled and/or abnormal division of cells that then invade and destroy the surrounding tissues. As used herein, “proliferating” and “proliferation” refer to cells undergoing mitosis. As used herein, “metastasis” refers to the distant spread of a malignant tumor from its sight of origin. Cancer cells may metastasize through the bloodstream, through the lymphatic system, across body cavities, or any combination thereof.

The term “carcinoma” refers to a malignant new growth made up of epithelial cells tending to infiltrate surrounding tissues, and to give rise to metastases.

The term “ameliorating” or “treating” means that the clinical signs and/or the symptoms associated with the cancer or melanoma are lessened as a result of the actions performed. The signs or symptoms to be monitored will be characteristic of a particular cancer or melanoma and will be well known to the skilled clinician, as will the methods for monitoring the signs and conditions. For example, the skilled clinician will know that the size or rate of growth of a tumor can monitored using a diagnostic imaging method typically used for the particular tumor (e.g., using ultrasound or magnetic resonance image (MRI) to monitor a tumor).

The term “anti-tumor effect” as used herein, refers to a biological effect which can be manifested by various means, including but not limited to, e.g., a decrease in tumor volume, a decrease in the number of tumor cells, a decrease in the number of metastases, an increase in life expectancy, decrease in tumor cell proliferation, decrease in tumor cell survival, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

The term “xenograft” as used herein, refers to a graft of tissue taken from a donor of one species and grafted into a recipient of another species.

### Description

The present invention relates to the discovery of a role for the nuclear receptor retinoid-related orphan receptor gamma (RORgamma) in tumor suppression. Expression of RORgamma results in gene activation within innate immune cells that trigger recognition and suppression of tumor cells.

### Compositions

The present invention provides a composition that triggers the expression of the nuclear receptor retinoid-related orphan receptor gamma (RORgamma), or a biologically functional fragment thereof, in a cell. As would be understood in the art, a biologically functional fragment is a portion of a full length sequence that retain the biological function of the full length sequence.

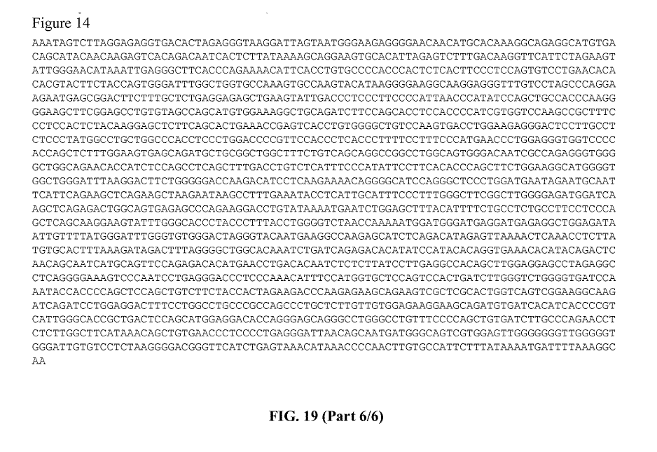

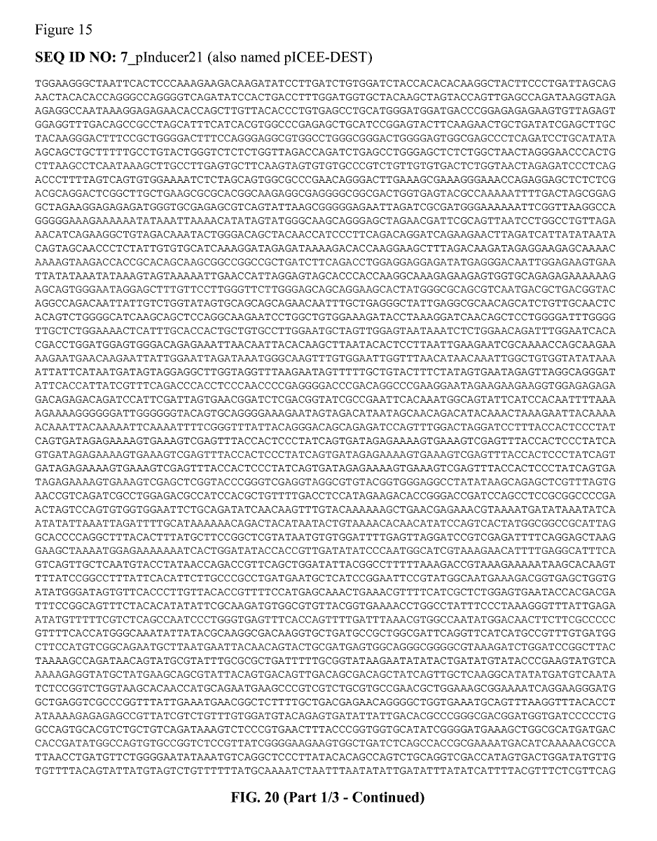

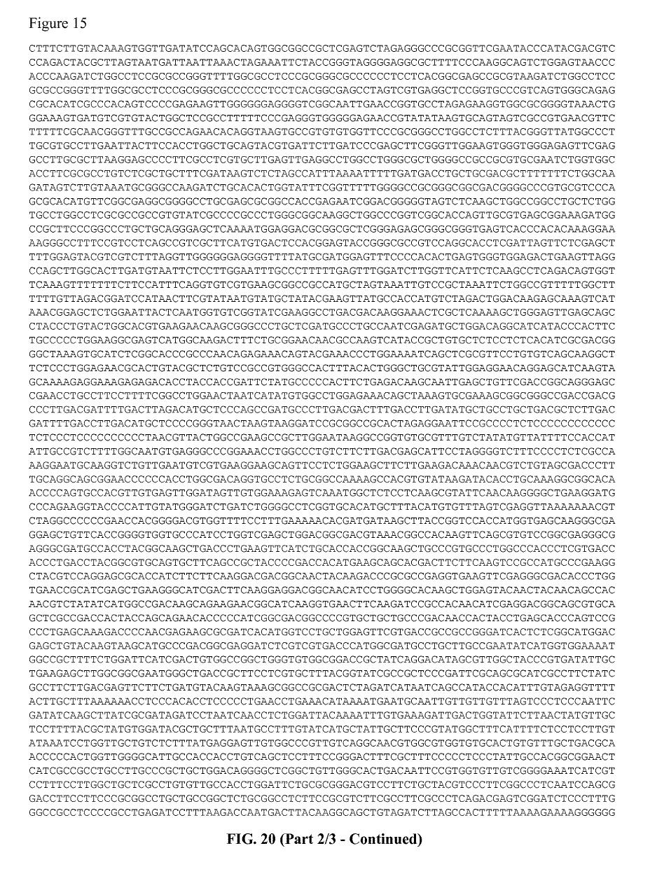

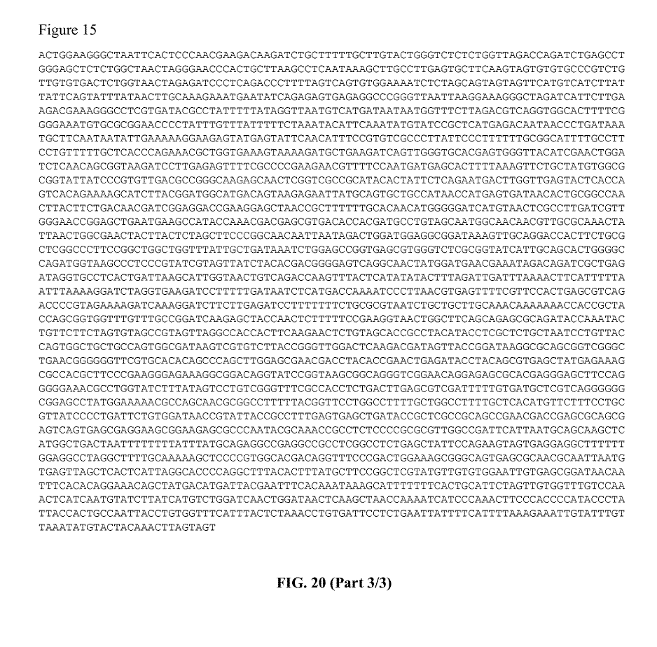

In one embodiment, the composition comprises an isolated nucleic acid comprising a sequence encoding a human RORgamma (RORC gene with SEQ ID NO: 6 (FIG. 19); RORC cDNA with SEQ ID NO: 9 (FIG. 15)), or a biologically functional fragment thereof. In one embodiment, the nucleic acid comprises a sequence encoding a mouse RORgamma (SEQ ID NO: 3), or a biologically functional fragment thereof. The isolated nucleic acid sequence encoding RORgamma can be obtained using various recombinant methods known in the art, such as, for instance by screening libraries from cells expressing the gene, by deriving the gene from a vector known to include the same, or by isolating directly from cells and tissues containing the same, using standard techniques. Alternatively, the RORC gene can be produced synthetically, rather than cloned. Further, the endogenous RORC locus may be modified in mammalian cells to alter expression levels and/or expression timing and/or alter the activity of RORC or a fragment of RORC.

In another embodiment, the composition comprises a recombinant RNA produced in vitro, or extracted from a prokaryotic or eukaryotic cells, which encodes RORgamma or a modified RORgamma (e.g. coding for fusion protein) or one of RORgamma's gene targets.

Further, the invention encompasses an isolated nucleic acid encoding a peptide having substantial homology to the peptides disclosed herein. Preferably, the nucleotide sequence of an isolated nucleic acid encoding a peptide of the invention is “substantially homologous”, that is, is about 60% homologous, more preferably about 70% homologous, even more preferably about 80% homologous, more preferably about 90% homologous, even more preferably, about 95% homologous, and even more preferably about 99% homologous to a nucleotide sequence of an isolated nucleic acid encoding a peptide of the invention.

In one embodiment, the composition comprises an isolated amino acid corresponding to a human RORgamma (SEQ ID NO: 1), or a biologically functional fragment thereof. In certain embodiments, the biologically functional fragment of RORgamma comprises a peptide that retains the function of full length RORgamma. In one embodiment, the amino acid comprises a mouse RORgamma (SEQ IDs NO: 4 and 5), or a biologically functional fragment thereof.

In one embodiment, RORgamma (being either from human or mouse origin, SEQ IDs NO: 1, 4 and 5 (FIG. 15; FIG. 18) is fused, through the N-terminal or C-terminal end, to either an activator (e.g. HSV VP16 domain) or repressor (e.g. KRAB domain) in order to enhance or inhibit expression of RORgamma's downstream targets. For example, RORgamma-VP16 fusion protein is utilized to enhance expression of scavenger receptors, and as a result induces a stronger homing and binding response of modified immune cells towards cancer cells. Another example would be to fuse a nuclear localization signal (nls) to RORgamma in order to enhance RORgamma's nuclear import and subsequent binding to recognition sites upstream of its target genes. RORgamma-nls fusion protein would be utilized to enhance expression of scavenger receptors, and as a result induces a stronger homing and binding response of modified immune cells towards cancer cells.

In a further embodiment, mutations that may or not increase RORgamma targeting and/or binding to target promoters may also be considered.

In another embodiment, the expression of a specific target or targets of RORgamma is enhanced or repressed through the use of custom DNA binding proteins. Non limiting examples include Zinc finger proteins, Tal-like effector nucleases (TALENs), and Cas9 mutated for loss of endonuclease and/or nicking activity coupled to a specific gRNA or set of gRNAs. The modified DNA binding protein(s) are introduced into immune stem cells and activate specific gene expression. For example, a modified Cas9 would be used to induce expression of the putative RORgamma scavenger receptor target, Scar2. Or a modified Cas9 protein would be used to repress putative RORgamma receptor target, CD148. Additionally, any version of a DNA binding protein that has the ability to modify the genome may be used to introduce a gene fragment with hRORgamma cDNA into cells of the innate immune system. This would involve genomic modification of the hRORgamma locus, or modification of the genomic DNA upstream or downstream hRORgamma via one of the various versions of homologous recombination. Additionally short, e.g. less than 1 kbp, native or chemically modified oligonucleotides could be employed to modify the endogenous hRORgamma locus. Alternatively, large genomic clones, e.g. 1 kbp or greater, with the intention of modifying the RORC locus or upstream or downstream activity of the RORC locus, may be introduced by a variety of means upon which homologous recombination or a similar phenomenon occurs.

The present invention also includes a vector in which the isolated nucleic acid of the present invention is inserted. The art is replete with suitable vectors that are useful in the present invention. In brief summary, the expression of natural or synthetic nucleic acids encoding RORgamma is typically achieved by operably linking a nucleic acid encoding the RORgamma or portions thereof to a promoter, and incorporating the construct into an expression vector. The vectors to be used are suitable for replication and, optionally, integration in eukaryotic cells. Typical vectors contain transcription and translation terminators, initiation sequences, and promoters useful for regulation of the expression of the desired nucleic acid sequence. The vectors of the present invention may also be used for nucleic acid standard gene delivery protocols. Methods for gene delivery are known in the art. See, e.g., U.S. Pat. Nos. 5,399,346, 5,580,859, 5,589,466, incorporated by reference herein in their entireties.

The isolated nucleic acid of the invention can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the vector may be provided to a cell in the form of a viral vector. Viral vector technology is well known in the art and is described, for example, in Sambrook et al. (4^{th} Edition, Molecular Cloning: A Laboratory Manual, Cold Spring Harbor Laboratory, New York, 2012), and in other virology and molecular biology manuals. Viruses, which are useful as vectors include, but are not limited to, retroviruses, adenoviruses, adeno-associated viruses, herpes viruses, Sindbis virus, gammaretrovirus and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used. For example, vectors derived from retroviruses such as the lentivirus are suitable tools to achieve long-term gene transfer since they allow long-term, stable integration of a transgene and its propagation in daughter cells (e.g. pinducer 21, SEQ ID NO: 7, Meerbrey et al., PNAS Vol. 108:9; 3665-3670, 2011). Lentiviral vectors have the added advantage over vectors derived from onco-retroviruses such as murine leukemia viruses in that they can transduce non-proliferating cells, such as hepatocytes. They also have the added advantage of low immunogenicity. In another embodiment, the composition includes a vector derived from an adeno-associated virus (AAV).

In some embodiments, the vector of this invention also includes conventional control elements which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

In one embodiment, a lymphoid specific promoter, or any lymphoid biased, promoter is used to drive expression in the stem cells of interest (e.g. innate immune cells of the lymphoid lineage). In another embodiment, a myeloid specific, or any myeloid biased, promoter is used to drive expression of the RORgamma transgene in the stem cells of interest. The nature the transfected cells of interest is described in details herein below. In a specific embodiment, the promoter is a neutrophil-specific promoter, such as but not limited to, the integrin alpha promoter CD11B (See FIG. 21, SEQ ID NO: 8; Shelley and Arnaout, Proc Natl Acad Sci USA. 1991; 88(23):10525-9) or the alpha defensin promoters DEFA1, DEFA2, DEFA3, and DEFA4.

In one embodiment, the expression of RORgamma is suppressed until the stem cells of interest have fully differentiated. In another embodiment, as the stem cells differentiate into neutrophils, macrophages or natural killer cells, the expression of the RORgamma transgene is induced.

In a further embodiment, the automatic induction of RORgamma allows for ex vivo differentiation and induction of the transgene before delivery to the patient, or transfer of modified stem cells to the patient after which natural or artificially induced differentiation results in production of the RORgamma protein.

In some instances, in order to assess the expression of a the gene of interest polypeptide of interest (e.g. the RORgamma) or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known in the art and may be prepared using known techniques or purchased commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are also well known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., Molecular Cloning: A Laboratory Manual, volumes 1-3 (4^{th} edition, Cold Spring Harbor Press, N Y 2012).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

In other instances, RORgamma is delivered via calcium phosphate transfection, nanoparticles, protein-conjugated DNA, peptide or peptide nuclear acid conjugated DNA, protein transduction (e.g. TAT or poly-arginine peptides) where the peptide or protein interact or are directly conjugated to the DNA encoding RORgamma.

### Cells

In one embodiment, stem cell lines are used in this invention. In another embodiment, the stem cell lines may be modified with RORgamma or may be modified to alter expression levels of RORgamma target genes (or their homologs).

In one embodiment, the stem cell lines belong to the innate immune cell family of cells. Non limiting examples of such cells are neutrophils, eosinophils, basophils, macrophages, dendritic cells, mast cells, mast cells, some B cells (B1 cells) and some T cells (e.g. natural killer T cells).

In yet another embodiment, the immune cells would be differentiated outside the body or allowed to differentiate within the host to, for example, natural killer cells.

In a further embodiment, the immune cells that complete differentiation either in vitro or in vivo are derived in one or more steps from an unrelated immune cells type. For example, induced pluripotent stem (iPS) cells could be differentiated from the patient or from cells intended to act as donor material for multiple patients. Those iPS cells, or iPS derived cells, would be modified to express RORgamma or a downstream RORgamma target gene or genes and then delivered to patients. Other examples include cells that may be transdifferentiated in one or more steps to myeloid precursors or mature myeloid cells.

### Sources of Myeloid Precursors Cells

Myeloid precursors cells can be separated from a complex mixture of cells by using reagents that specifically recognize markers on the cell surface, including CDw127 (IL-7 receptor α); CD117 (c-kit) protein, and a cocktail of markers expressed on lineage committed cells. Myeloid precursors cells of the invention are characterized on the basis of a positive expression of the antigen CD34 (CD34⁺). As general example, but not limited to, these cells are also characterized by the expression of Fcγ receptor (FcγR), IL-7Rα negative, sca-1 negative, lineage negative, and c-kit high.

In one embodiment, methods for enrichment of myeloid precursor cells are provided. The enriched cell population will usually have at least about 90% cells of the selected phenotype, more usually at least 95% cells of the selected phenotype. The subject cell populations are separated from other cells, e.g. hematopoietic cells, on the basis of specific markers, which are identified with affinity reagents, e.g. monoclonal antibodies. The myeloid precursors cells can be isolated from any source of hematopoietic progenitor cells, which may be fetal, neonatal, juvenile or adult, including bone marrow, spleen, liver, umbilical cord blood, peripheral blood, mobilized peripheral blood, yolk sac, etc. For autologous or allogeneic transplantation, bone marrow and mobilized peripheral blood are preferred starting materials. For peripheral blood, progenitor cells are mobilized from the marrow compartment into the peripheral bloodstream after treatment with chemotherapy; G-CSF or GM-CSF, or both. A number of single and combination chemotherapeutic agents have been used to mobilize peripheral blood progenitor cells (PBPCs). In administering these agents, a balance must be found in all cases between effective PBPC mobilization and possible damage to the hematopoietic stem cell pool and overall patient tolerance. Paclitaxel has been found to effectively mobilize PBPCs without damaging the stem cell pool. A review of peripheral blood stem cells may be found in Shpall et al. (1997) Annu Rev Med 48:241-251, and the characterization of stem cell mobilization in Moog et al. (1998) Ann Hematol 77(4):143-7. As an alternative source of cells, hematopoietic stem cells, as described in U.S. Pat. No. 5,061,620, issued on Oct. 29, 1991; and U.S. Pat. No. 5,087,570, issued Feb. 11, 1992, may be cultured in vivo or in vitro to provide a source of cells.

In another embodiment, the myeloid precursors cells may be obtained from any mammalian species, e.g. equine, bovine, porcine, canine, feline, rodent, e.g. mice, rats, hamster, primate, etc., particularly human. The tissue may be obtained by biopsy or aphoresis from a live donor, or obtained from a dead or dying donor within about 48 hours of death, or freshly frozen tissue, tissue frozen within about 12 hours of death and maintained at below about −20° C., usually at about liquid nitrogen temperature (−180° C.) indefinitely. The myeloid precursors cells are characterized by their expression of cell surface markers. For several of these markers, the expression is low or intermediate in level. While it is commonplace to refer to cells as “positive” or “negative” for a particular marker, actual expression levels are a quantitative trait. The number of molecules on the cell surface can vary by several logs, yet still be characterized as “positive”. Characterization of the level of staining permits subtle distinctions between cell populations.

In yet another embodiment, the staining intensity of cells can be monitored by flow cytometry, where lasers detect the quantitative levels of fluorochrome (which is proportional to the amount of cell surface antigen bound by the antibodies). Flow cytometry, or FACS, can also be used to separate cell populations based on the intensity of antibody staining, as well as other parameters such as cell size and light scatter. Although the absolute level of staining may differ with a particular fluorochrome and antibody preparation, the data can be normalized to a control.

In a further embodiment, the myeloid precursor cells are separated from a complex mixture of cells by techniques that enrich for cells having the above characteristics. For isolation of cells from tissue, an appropriate solution may be used for dispersion or suspension. Such solution will generally be a balanced salt solution, e.g. normal saline, PBS, Hank s balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

In one embodiment, separation of myeloid precursors cell populations can use affinity separation to provide a substantially pure population. Techniques for affinity separation may include magnetic separation, using antibody-coated magnetic beads, affinity chromatography, cytotoxic agents joined to a monoclonal antibody or used in conjunction with a monoclonal antibody, e.g. complement and cytotoxins, and “panning” with antibody attached to a solid matrix, eg. plate, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide). Any technique may be employed which is not unduly detrimental to the viability of the selected cells.

In another embodiment, the affinity reagents may be specific receptors or ligands for the cell surface molecules indicated above. In addition to antibody reagents, peptide-MHC antigen and T cell receptor pairs may be used; peptide ligands and receptor; effector and receptor molecules, and the like. Antibodies and T cell receptors may be monoclonal or polyclonal, and may be produced by transgenic animals, immunized animals, immortalized human or animal B-cells, cells transfected with DNA vectors encoding the antibody or T cell receptor, etc. The details of the preparation of antibodies and their suitability for use as specific binding members are well-known to those skilled in the art.

In yet another embodiment, antibodies are used as affinity reagents. Conveniently, these antibodies are conjugated with a label for use in separation. Labels include magnetic beads, which allow for direct separation, biotin, which can be removed with avidin or streptavidin bound to a support, fluorochromes, which can be used with a fluorescence activated cell sorter, or the like, to allow for ease of separation of the particular cell type. Fluorochromes that find use include phycobiliproteins, e.g. phycoerythrin and allophycocyanins, fluorescein and Texas red. Frequently each antibody is labeled with a different fluorochrome, to permit independent sorting for each marker. The antibodies are added to a suspension of cells, and incubated for a period of time sufficient to bind the available cell surface antigens. The incubation will usually be at least about 5 minutes and usually less than about 30 minutes. It is desirable to have a sufficient concentration of antibodies in the reaction mixture, such that the efficiency of the separation is not limited by lack of antibody. The appropriate concentration is determined by titration. The medium in which the cells are separated will be any medium which maintains the viability of the cells. A preferred medium is phosphate buffered saline containing from 0.1 to 0.5% BSA. Various media are commercially available and may be used according to the nature of the cells, including Dulbecco s Modified Eagle Medium (dMEM), Hank's Basic Salt Solution (HBSS), Dulbecco s phosphate buffered saline (dPBS), RPMI, Iscove s medium, PBS with 5 mM EDTA, etc., frequently supplemented with fetal calf serum, BSA, HSA, etc. The labeled cells are then separated as to the expression of c-kit, IL-7Rα, and lin panel. The selected population is c-kit high, lin negative, IL-7Rα negative. The cell population could also divided into subsets based on expression of FcγR and CD34. The separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum.

In other embodiments, the cells may be incubated on a rotator for varying lengths of time at varying speeds at either 2-10° C. or at room temperature. In further embodiments, cells can also be frozen after a washing step. Methods of controlled freezing may be used as well as uncontrolled freezing immediately at −20° C. or in liquid nitrogen. In certain embodiments, cryopreserved cells are thawed and washed and allowed to rest for one hour at room temperature prior to activation using the methods of the present invention.

In one embodiment, myeloid precursors modified with inducible hRORgamma are cultured in maintenance media until differentiation into neutrophils is desired. The myeloid precursors are then switched from maintenance media to media used to induce differentiation, as well as doxycycline to induce the expression of the hRORgamma transgene. The differentiated neutrophils, expressing the hRORgamma transgene, are then delivered intravenously to the patient (e.g. with non-small cell lung cancer). The hRORgamma transgenic neutrophils then home in on, bind to and inhibit growth of or induce cell death of the target cancer cells.

In another embodiment, precursor cells are maintained in a stem-like state through the use of an immortalization agent. For example, inducible hTERT or inducible Hoxa9 or HoxB8 (e.g. delivered via Lentivirus or protein transduction) or other protein with cell lifespan conferring ability may be used to immortalize myeloid precursors which normally would have a limited lifespan. In doing so, a myeloid line with limited lifespan could be cultured indefinitely in order to more easily industrialize the process of producing anticancer immune cells, producing a universal ‘bank’ of donor cells for patients. The universal bank of cells could be modified with factors such as RORgamma, and the expanded, but would not need to be rederived as long as cells and gene expression patterns and genomic sequence remained consistent as determined by methods known to those skilled in the art.

In yet another embodiment, RORgamma or RORgamma target genes are upregulated or repressed in cells which have been blocked from further cell division through the use of a chemical treatment or radiation. As a result, the donor cells would not post a threat of potentially harming the patient through uncontrolled growth if for instance a retroviral insertion resulted in the upregulation of a cell growth and/or cell division inducing protein.

In another embodiment, RORgamma or one or more of its downstream targets may be altered in cells which have low naturally occurring or artificially reduced levels of MHC class molecules in order to allow for universal transplantation.

### Diagnosis and Treatment

In one embodiment, the invention relates to a method of diagnosing a cancer or a predisposition for developing a cancer or a metastasis in a subject. The method comprises determining the expression level of RORgamma gene in immune cells (e.g. neutrophils) in a biological sample from the subject, wherein an increase in the expression level of RORgamma as compared with a normal control level of RORgamma expression is an indication that the subject has cancer or has a predisposition for developing a cancer or metastasis.

In another embodiment, the invention relates to a method for determining the efficacy of immunotherapy treatment for treating cancer in a subject in need thereof. The method comprises determining the expression level of RORgamma gene in neutrophil cells in a biological sample from the subject, wherein an increase in the expression level of RORgamma as compared with the expression level of RORgamma in a normal control is an indication that immunotherapy treatment will be effective. In some aspects of the invention, treatment of cancer may include the treatment of solid tumors or the treatment of metastasis. Metastasis is a form of cancer wherein the transformed or malignant cells are traveling and spreading the cancer from one site to another. Such cancers include cancers of the skin, breast, brain, cervix, testes, etc. More particularly, cancers may include, but are not limited to the following organs or systems: cardiac, lung, gastrointestinal, genitourinary tract, liver, bone, nervous system, gynecological, hematologic, skin, and adrenal glands. Furthermore, the methods herein can be used for treating gliomas (Schwannoma, glioblastoma, astrocytoma), neuroblastoma, pheochromocytoma, paraganlioma, meningioma, adrenalcortical carcinoma, kidney cancer, vascular cancer of various types, osteoblastic osteocarcinoma, prostate cancer, ovarian cancer, uterine leiomyomas, salivary gland cancer, choroid plexus carcinoma, mammary cancer, pancreatic cancer, colon cancer, and megakaryoblastic leukemia. Skin cancer includes malignant melanoma, basal cell carcinoma, squamous cell carcinoma, Karposi's sarcoma, moles dysplastic nevi, lipoma, angioma, dermatofibroma, keloids, and psoriasis.

In one embodiment, the invention comprises a method of providing anti-tumor immunity and for stimulating innate immune response by administering the to the subject a viral vector comprising a nucleic acid sequence encoding RORgamma wherein expression of RORgamma is under the control of a neutrophil specific promoter.

In another embodiment, the immune stimulation is achieved by administering the to the subject a cell (e.g. CD34⁺ cell) comprising a viral vector comprising a nucleic acid sequence encoding RORgamma wherein expression of RORgamma is under the control of a neutrophil specific promoter.

In one embodiment, the subject suitable for human RORgamma (hRORgamma) treatment may include the following: human subjects or any wild or domesticated mammal needing to undergo cancer treatment. In the case of mammals other than human cancer patients, the appropriate homologs of hRORgamma (SEQ ID NO: 1) would be utilized—e.g. canine RORgamma for domesticated dogs undergoing immune modification for cancer therapy or prevention.

In another embodiment, RORgamma product or RORgamma's target genes may be upregulated indirectly within a desired cell population. Indirect activation could include—e.g. ectopic expression of a transcription factor, kinase, phosphatase or any protein, peptide or non-translated macro or microRNA that affects hRORgamma expression. The invention could also involve artificial zinc finger proteins or other genomic DNA binding proteins artificially produced to target the key downstream targets of hRORgamma to confer a similar cancer killing activity upon cells of the innate immune system.

In yet another embodiment, a kill switch is incorporated into the immune cells modified with RORgamma or RORgamma target genes in order to protect the patient from any potential out of control growth of the donor cells. For example, the continual production of the prodrug converting enzyme thymidine kinase (TK) may be engineered into the donor immune cells by methods known to those trained in the art. Cells producing TK in turn are eliminated upon administration of the prodrug Ganciclovir. This approach is useful in cases, as an example, where the immune cells have accrued genetic damage resulting in out of control growth and must be eliminated from the patient.

In a further embodiment, the hRORgamma-based treatment may be combined with other treatments so long as the parallel treatment does not interfere with hRORgamma expression and/or function or the expression and/or function of the effective downstream targets of RORgamma, or cause a depletion of neutrophils or other immune cell types that have been modified to home in on (or better home in on) and block and/or cause the death of cancer cells.

In one embodiment, the delivery of a composition comprising RORgamma could be used for cancer prevention in a subject in need thereof.

In another embodiment, patients who are determined to be susceptible to cancer via genetic makeup or lifestyle (e.g. smokers) may receive modified hematopoietic cells with long lifespan (e.g. one year lifespan) to act as a cancer prevention measure. Non limiting examples are early stage hematopoietic stem cells with multipotent differentiation potential, or adult macrophages that no longer divide but retain a long lifespan. In this instance, the cells of interest will have a longer lifespan than neutrophils. These cells are modified for production of RORgamma or RORgamma downstream targets such as scavenger receptor (e.g. homologs of Scart2) or inhibition of CD148 or CD148's effectors, and then are delivered to the patient at risk for developing a cancer.

### Administration/Dosing

The regimen of administration may affect what constitutes an effective amount. For example, the therapeutic formulations may be administered to the patient either prior to or after a surgical intervention related to cancer, or shortly after the patient was diagnosed with cancer. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat cancer in the patient. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 0.01 and 50 mg/kg of body weight/per day. One of ordinary skill in the art would be able to study the relevant factors and make the determination regarding the effective amount of the therapeutic compound without undue experimentation.

The compound can be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon any number of factors, such as, but not limited to, the type and severity of the disease being treated, and the type and age of the animal.

### Routes of Administration

One skilled in the art will recognize that although more than one route can be used for administration, a particular route can provide a more immediate and more effective reaction than another route.

Routes of administration of any of the compositions of the invention include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration. Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Control Standard Amount of Expression of the Gene of Interest (RORgamma)

The method of the invention includes comparing a measured amount of expression of RORgamma in neutrophil cells in a biological sample from a subject to a control amount (i.e. the reference) of expression of RORgamma.

In one embodiment, the standard control level of expression of RORgamma may be obtained by measuring the expression level of RORgamma in innate immune cells in a healthy subject (e.g. in neutrophils). Preferably, the healthy subject is a subject of similar age, gender and race and has never been diagnosed with any type of sever disease particularly any type of cancer.

In another embodiment, the control amount of expression of RORgamma is a value for expression of RORgamma that is accepted in the art. This reference value can be baseline value calculated for a group of subjects based on the average or mean values of RORgamma expression by applying standard statistically methods

In one embodiment, the expression level is determined by a method selected from the group consisting of detecting mRNA of the gene, detecting a protein encoded by the gene, and detecting a biological activity of the protein encoded by the gene.

In certain aspects of the present invention, the expression level of RORgamma is determined in a sample from a subject. The sample preferably includes tumor cells, any fluid from the surrounding of tumor cells (i.e., leukemic blood, tumor tissue, etc. . . . ) or any fluid that is in physiological contact or proximity with the tumor, or any other body fluid in addition to those recited herein should also be considered to be included in the invention. In a specific embodiment, the sample preferably includes innate immune cells (e.g. neutrophils).

### Methods of Measurement

Any method known to those in the art can be employed for determining the level of RORgamma expression. For example, a microarray can be used. Microarrays are known in the art and consist of a surface to which probes that correspond in sequence to gene products (e.g. mRNAs, polypeptides, fragments thereof etc.) can be specifically hybridized or bound to a known position. To detect at least one gene of interest, a hybridization sample is formed by contacting the test sample with at least one nucleic acid probe. A preferred probe for detecting RORgamma is a labeled nucleic acid probe capable of hybridizing to RORgamma mRNA. The nucleic acid probe can be, for example, a full-length nucleic acid molecule, or a portion thereof, such as an oligonucleotide of at least 10, 15, or 20 nucleotides in length and sufficient to specifically hybridize under stringent conditions to the appropriate target. The hybridization sample is maintained under conditions which are sufficient to allow specific hybridization of the nucleic acid probe to a target of interest. Specific hybridization can be performed under high stringency conditions or moderate stringency conditions, as appropriate. In a preferred embodiment, the hybridization conditions for specific hybridization are high stringency. Specific hybridization, if present, is then detected using standard methods. If specific hybridization occurs between the nucleic acid probe and a gene in the test sample, the sequence that is present in the nucleic acid probe is also present in the mRNA of the subject. More than one nucleic acid probe can also be used. Hybridization intensity data detected by the scanner are automatically acquired and processed by the Affymetrix Microarray Suite (MASS) software. Raw data is normalized to expression levels using a target intensity of 150. An alternate method to measure mRNA expression profiles of a small number of different genes is by e.g. either classical TaqMan® Gene Expression Assays or TaqMan® Low Density Array—micro fluidic cards (Applied Biosystems). Particularly, this invention preferably utilizes a qPCR system. Non-limiting examples include commercial kits such as the PrimePCRPathways® commercially available from Bio-rad (Berkley, Calif.).

The transcriptional state of a sample, particularly mRNAs, may also be measured by other nucleic acid expression technologies known in the art. mRNA can be isolated from the sample using any method known to those in the art. Non-limiting examples include commercial kits, such as the RNeasy® commercially available from Qiagen (Netherlands) or the Mini Kit the TRI Reagent® commercially available from Molecular Research Center, Inc. (Cincinnati, Ohio), can be used to isolate RNA. Generally, the isolated mRNA may be amplified using methods known in the art. Amplification systems utilizing, for example, PCR or RT-PCR methodologies are known to those skilled in the art. For a general overview of amplification technology, see, for example, Dieffenbach et al., PCR Primer: A Laboratory Manual, Cold Spring Harbor Laboratory Press, New York (1995).

Another accurate method for profiling mRNA expression can the use of Next Generation Sequencing (NGS) including first, second, third as well as subsequent Next Generations Sequencing technologies.

In other aspects of the present invention, determining the amount or detecting the biological activity of a peptide, polypeptide can be achieved by all known means in the art for determining the amount of a peptide or polypeptide in a sample. These means comprise immunoassay devices and methods which may utilize labeled molecules in various sandwich, competition, or other assay formats. Such assays will develop a signal which is indicative for the presence or absence of the peptide or polypeptide. Moreover, the signal strength can, preferably, be correlated directly or indirectly (e.g. reverse-proportional) to the amount of polypeptide present in a sample. Further suitable methods comprise measuring a physical or chemical property specific for the peptide or polypeptide such as its precise molecular mass or NMR spectrum. Said methods comprise, preferably, biosensors, optical devices coupled to immunoassays, biochips, analytical devices such as mass-spectrometers, NMR-analyzers, or chromatography devices. Further, methods include micro-plate ELISA-based methods, fully-automated or robotic immunoassays (available for example on Elecsys™ analyzers), CBA (an enzymatic Cobalt Binding Assay, available for example on Roche-Hitachi™ analyzers), and latex agglutination assays (available for example on Roche-Hitachi™ analyzers).

### Kit

The invention includes a set of preferred antibodies, either labeled (e.g., fluorescer, quencher, etc.) or unlabeled, that are useful for the detection of at least RORgamma.

In certain embodiments, a kit is provided. Commercially available kits for use in these methods are, in view of this specification, known to those of skill in the art. In general, kits will comprise a detection reagent that is suitable for detecting the presence of a polypeptide or nucleic acid, or mRNA of interest.

In another embodiment, the kit includes a panel of probe sets or antibodies. Probe sets are designed to detect the level of RORgamma in innate immune cells and provide information about anti-tumor immunity. Probe sets are particularly useful because they are smaller and cheaper than probe sets that are intended to detect as many peptides as possible in a particular genome. In the present invention, the probe sets are targeted at the detection of polypeptides that are informative about cancer incidence. Probe sets may also comprise a large or small number of probes that detect peptides that are not informative about cancer. Such probes are useful as controls and for normalization (e.g., spiked-in markers). Probe sets may be a dry mixture or a mixture in solution. In some embodiments, probe sets can be affixed to a solid substrate to form an array of probes. The probes may be antibodies, or nucleic acids (e.g., DNA, RNA, chemically modified forms of DNA and RNA), LNAs (Locked nucleic acids), or PNAs (Peptide nucleic acids), or any other polymeric compound capable of specifically interacting with the peptides or nucleic acid sequences of interest.

In yet another embodiment, the kit includes instructions for use that comprise detecting the level of RORgamma in innate immune cells in a sample from a mammal in need thereof, providing indication on presence or absence of anti-tumor immunity, and providing recommendation of whether or not anti-tumor immunity treatment, which comprises administering to the mammal a composition comprising a viral a nucleic acid sequence encoding RORgamma wherein expression of RORgamma is under the control of a neutrophil promoter, should be administered, modified or terminated in the mammal.

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

The materials and methods employed in the experiments disclosed herein are now described.

### Materials and Methods

RNA-Seq.

The RNA-Seq library preparation protocol used for SR/CR differential display was based on the generic RNAseq protocol know in the art (TruSeq protocol, Illumina, San Diego, Calif.). Briefly, the protocol includes the steps below:

Isolation of total RNA and microRNAs using mirVana RNA Isolation Kit.

Purification of polyA using MicroPoly(A) Purist Kit from Ambion.

RNA Fragmentation.

Double-Stranded cDNA synthesis-Invitrogen (Cat#11917-020).

End Repair using End-It DNA End Repair Kit Epicentre (Cat# ER0720).

Addition of “A′ base to 3′ Ends.

Adapter ligation: Use LigaFast from Promega (Cat# M8221) and the PE Adapter Oligo Mix (part#1001782) from Illumina.

PCR to Amplify Library and Size Selection: Use Phusion DNA polymerase, NEB (Cat# F-531) and Illumina primers.

Cell Killing Assays.

Mouse RORgamma cDNA (mouse RORC nucleic acid, SEQ ID NO: 3, FIG. 17) contained in a Gateway System Entry vector (Life Technologies) was transferred into the Mach7 Yale Lentiviral vector (SEQ ID NO: 2, FIG. 16) via LR recombination reaction using LR Clonase (Life Technologies). Treated samples were transformed into chemically competent Top10 cells (Life Technologies) via heat shock transformation and selected on LB+100 ug/mL ampicillin plates overnight at 30° C. Single colonies were picked and grown in 2 mL of LB+100 ug/mL ampicillin overnight at 30° C. with vigorous shaking. Bacteria were pelleted and DNA extracted using Qiagen Qiaprep spin miniprep kit. Approximately 0.5-1.0 micrograms of DNA of each preparation was digested with restriction enzyme BsrGI (New England Biolabs) for two hours at 37° C. Entire digests were loaded onto 1% w/v TAE agarose gels containing 0.5 ug/mL ethidium bromide and run at 60V at constant voltage for several hours. Two clones in which the correct insert size and backbone correlating to the Mach7 Lentiviral vector were chosen for production of infectious Lentiviral particles.

Viral packaging of the Mach7-mRORgamma constructs was carried out through the use of transient Lentiviral packaging protocol. The HEK293 derivative cell line VNRC was plated the night prior to transfection at ˜5×10E7 cells/well in 10 cm tissue culture dishes in DMEM cell culture medium (Life Technologies) containing 10% heat inactivated fetal bovine serum (Life Technologies) and lacking antibiotics. The following day cells were transfected if they had reach confluence of ˜90%. The lentiviral packaging mix consisted of three vectors in the following ratios: 2.5 ug of Mach7-mRORgamma, 3.3 ug of the packaging plasmid pCMVRDelta8.91 and 0.8 ug of the envelope plasmid pMD2.G. The Lentiviral packaging mix was diluted in serum free DMEM and mixed with an equal volume of serum free DEME containing 14 microliters of Lipofectamine 2000 (Life Technologies). Mixture was incubated at room temperature for 30 minutes before being added dropwise to VNRC cells plated a day earlier. VNRC cells were incubated in humidified chambers with 5% CO2 and left undisturbed for 48 hours. At 48 hrs post-transfection cell culture supernatant was collected, centrifuged at room temperature for 5 minutes at 300×g to remove cell debris and filtered through 0.45 micron PVDF syringe filters to remove any remaining cell debris.

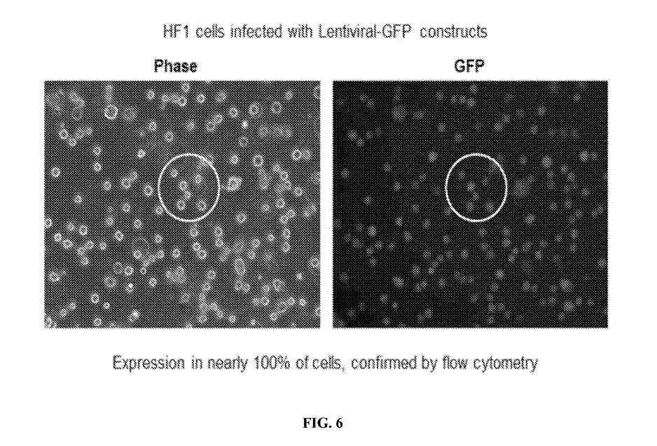

Target cell lines for infection were HF-1 myeloid precursor cells. HF-1 precursors were maintain in a precursor state by continued culture in the presence of recombinant mouse GM-CSF. 5 mL of undiluted viral supernatant per clone was added to 2 million HF-1 one cells suspended in 1 mL of culture media (IMDM, 15% heat-inactivated fetal bovine serum and 2.5 ug/mL mouse GM-CSF). To aid in infection, hexa dimethrine bromide (Polybrene, Sigma Aldrich) was added to the infection mixture at 6 ug/mL. Samples were then centrifuged at 1000×g for one hour to aid in viral particle contact and infection of HF-1 cells (a process termed ‘spinfection’). Following spinfection, HF-1 treated samples were resuspended in existing media and topped off with 5 mL of additional GM-CSF media before transfer to T25 vented tissue culture flasks and incubated at 37 C overnight. The following day the infected HF-1 cells were pelleted, and resuspended in fresh HF1 culture media. 48 hrs post-infection blasticidin was added to cell culture at a final concentration of 15 ug/mL to select for cell clones that had stably integrated the Mach7-mRORgamma construct. As a control, viral production was conducted in parallel with Mach7-red fluorescent protein (mRFP) to ensure viral infection and selections were effective. Similarly, Mach7-green fluorescent protein (GFP) was utilized as a control and upon examination with fluorescence microscopy was determined to be expressed in nearly all cells (FIG. 6). Mach7-mRORgamma and controls were further expanded for cell killing assays.

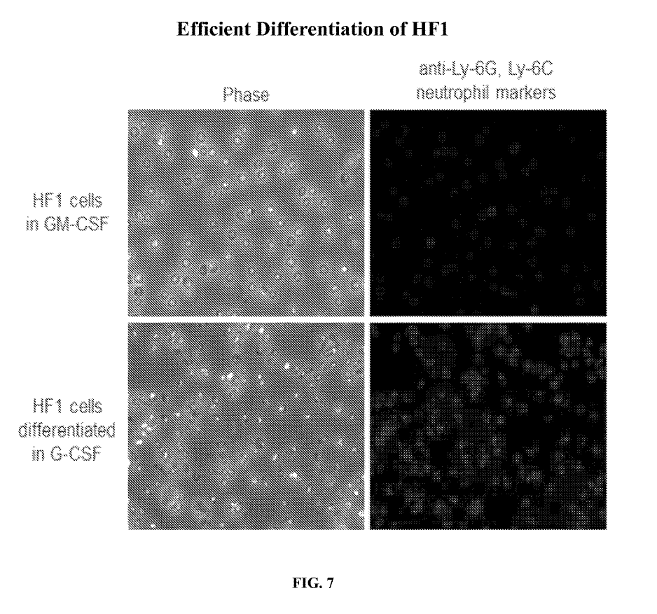

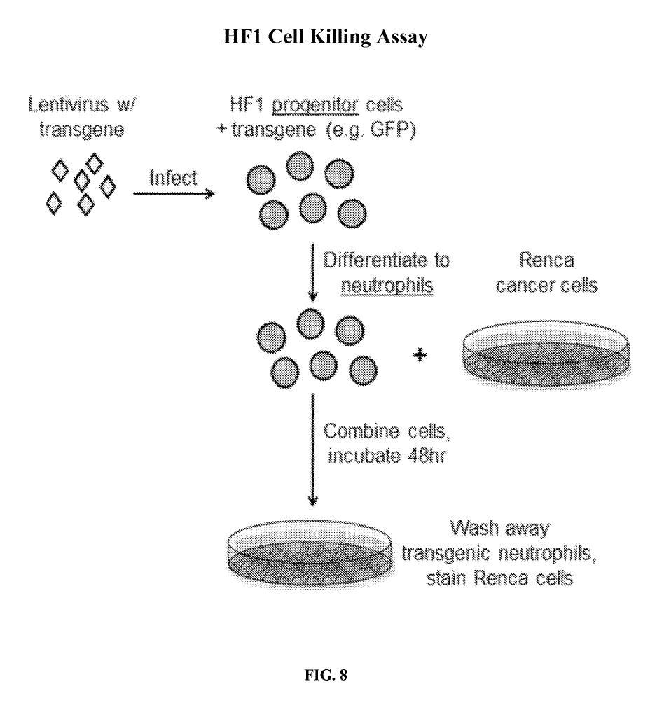

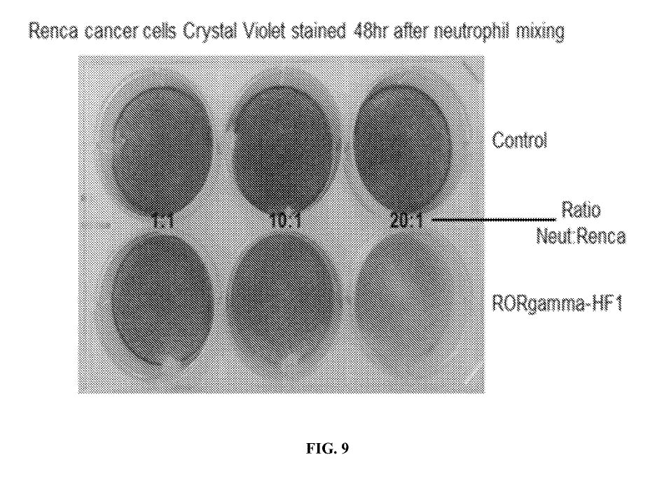

The renal cancer cell line Renca (American Type Culture Collection) was chosen to determine if any cancer cell killing ability had been conferred upon HF-1 derived neutrophils. Renca cells are adherent and would allow for straightforward visualization of cell death vs the HF-1 derived neutrophils which are a suspension cell line (FIG. 8). HF1 cells (unaltered or transgenic) were differentiated by washing cells to remove GM-CSF from the media and were supplemented with 50 ng/mL G-CSF in order to promote differentiation to the neutrophil lineage. Success of differentiation was determined by positive immunostaining with the neutrophil-specific antibody NIMP-R14 (Abcam) on cells that had been cultured in differentiation media for 72 hours (FIG. 7). Once it was determined that the HF-1 derived lines could be efficiently differentiated, the various cell lines were expanded in HF-1 culture media. Cancer killing assays were executed by plating Renca cells in DMEM+10% fetal bovine serum the night before the assay at a density of 3×10E5 cells per 6-well well. The following morning the HF-1 cell lines that had been differentiating in G-CSF media for 72 hours were pelleted, and resuspended in fresh G-CSF media. HF-1 lines at various ratios were then added to the wells of plated Renca cells (Renca having reached ˜25-35% confluence) and the coculture was incubated at 37 C for 48 hours. The cell killing assay came to completion by aspirating the cell culture supernatant to remove neutrophils and adding 2 mL per well of 1% w/v Crystal Violet in 50% ethanol in water to both fix the Renca cells and stain them for easy visualization (FIG. 9). It was consistently determined that there was a substantial cell killing effect of Renca cells co-incubated with HF-1 cells modified with Mach7-mRORgamma. Control neutrophils derived from HF-1 cells modified with either RFP or closely related mRORgamma splice form mRORgamma-t or ribonuclear RNA snora69 showed no killing or slight toxicity at the highest ratio of 20:1 neutrophils:Renca cells (FIG. 9).

### Example 1: Transcriptome Analysis Revealed Candidate Genes Significantly Upregulated in the SR/CR Mice

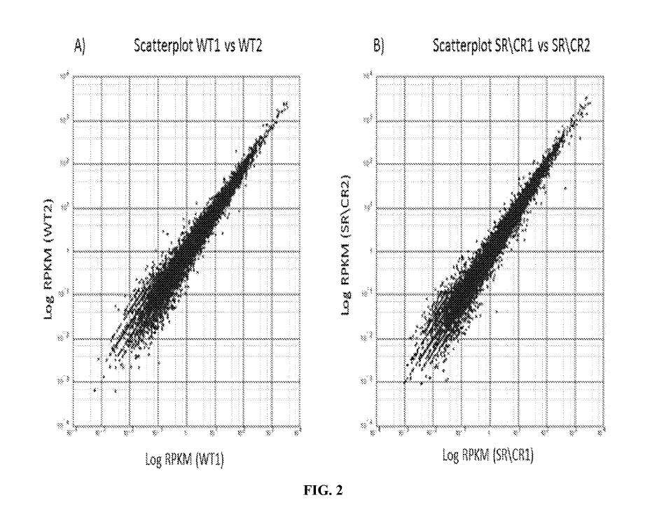

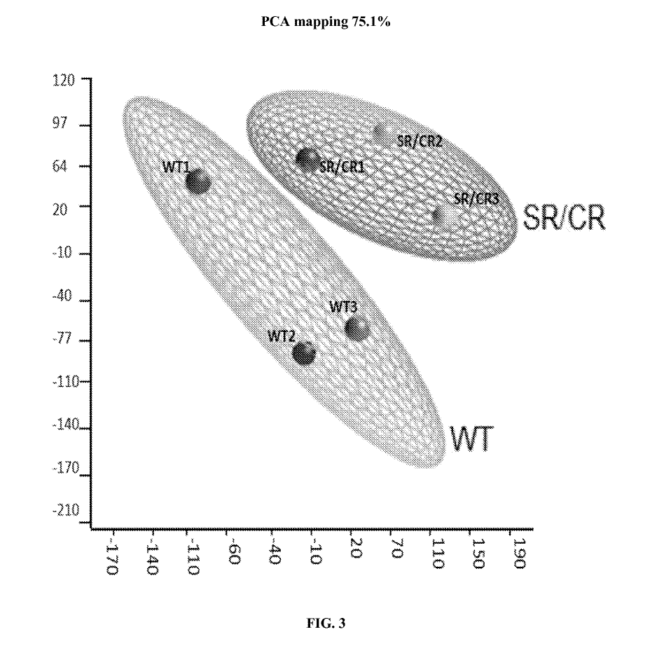

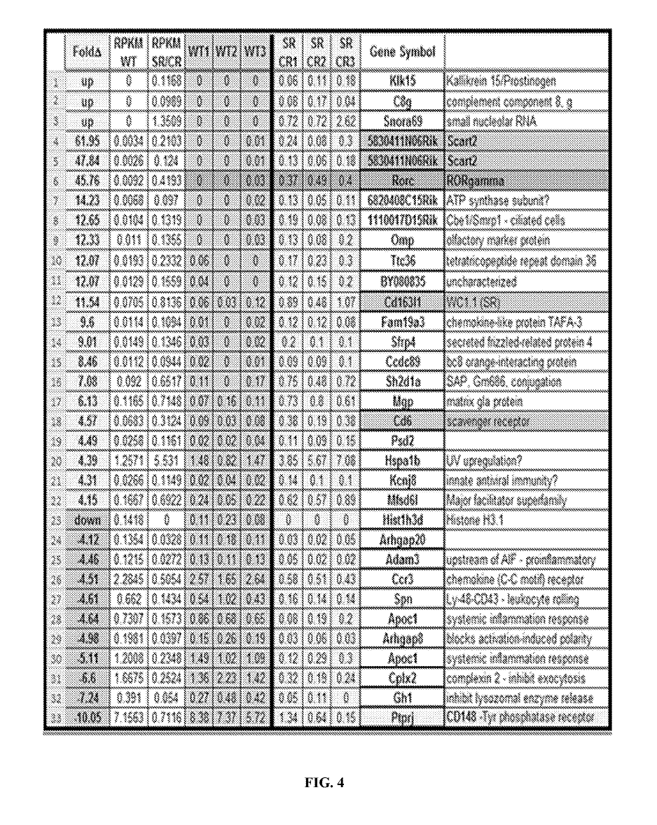

Samples consisted of the following: ˜20 million neutrophils from naïve SR/CR mice (previously unchallenged with cancer cells) were compared to neutrophils from control littermates. Information was recorded on which samples belonged to i) cancer resistant mice and ii) wild type control mice. The transcriptome of three SR/CR neutrophil samples and three control littermate neutrophil samples were assessed via next generation sequencing (RNA-Seq using TruSeq protocol, Illumina, San Diego, Calif.), (FIGS. 2A-2B). 60 million reads were obtained from each sample (FIG. 1). The computational analysis highlighted candidate genes significantly upregulated in the SR/CR mice (FIG. 3). For instance, these results showed a significant upregulation in the Scavenger receptors, an altered expression of a receptor involved in chemotactic response, and a 45 fold upregulation in the nuclear receptor RORgamma. Based on these results, the factors with the most dramatic upregulation in SR/CR mice were analyzed via ectopic expression in a cell culture model. A number of the top scoring factors, based on fold increase and statistically significant P value (FIG. 4), were incorporated into a lentiviral expression vector. As an example, these factors consist of, but are not limited to, RORgamma, the splice variant RORgamma-t and snora69.

### Example 2: Delivery and Expression of Mouse RORgamma Confers Cancer Killing Activity to In Vitro Derived Neutrophils

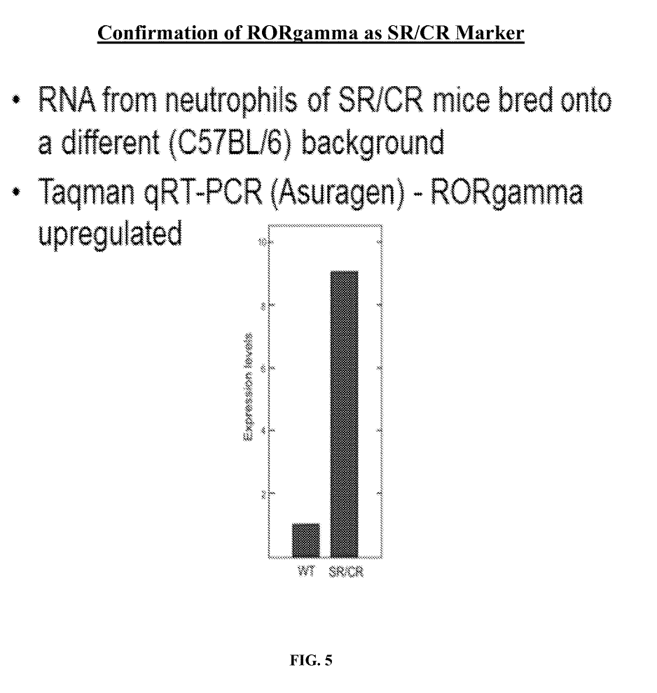

Lentiviral constructs were used to infect HF1-Hoxa9 myeloid precursor cells. The HF1-Hoxa9 cells were maintained in a precursor state as long as they were cultured in the presence of GM-CSF factor. Upon withdrawal of GM-CSF and culture in the presence of G-CSF, nearly 100% of the cells differentiated into neutrophils. These transgenic, differentiated neutrophils were used in cell killing assays where the neutrophils (suspension cells) added to wells containing adherent Renca cancer cells—after 48 hrs Crystal Violet staining would determine if there was any loss/clearing of the adherent cancer cells (FIG. 9). The only factor that had an impact on the adherent cancer cells (slowed growth and/or resulted in cell death of the adherent cancer cell line) was RORgamma. It was observed in the RNA-Seq data analysis that RORgamma was the only transcription factor that had both a statistically significant P-value and a very significant upregulation (45 fold increase) compared to wild type neutrophil controls. Additional validation of the results was done via Taqman qRT-PCR (FIG. 5). These results confirmed that there is significant upregulation of RORgamma in neutrophils from cancer resistant SR/CR mice compared to controls. The results also confirmed that RORgamma could be used as a marker for determining SR/CR mice or possibly as a marker correlating to anticancer killing activity of human neutrophils.

The expression profile of the SR/CR mice, combined with the functional cell biology assays performed with RORgamma, indicate that RORgamma is the key factor that has given SR/CR mice the remarkable ability to resistant cancer and even regress primary tumors in an aggressive model of prostate cancer. This information has the potential to revolutionize cancer therapy by not only allowing the modification of mature neutrophils or stem cells to attack cancer cells, but also provide a means to protect those in generally good health from getting cancer (similar in concept to a vaccine).

### Example 3: Induced Expression of Mouse RORgamma Confers Cancer Killing Activity to in Vitro Derived Neutrophils

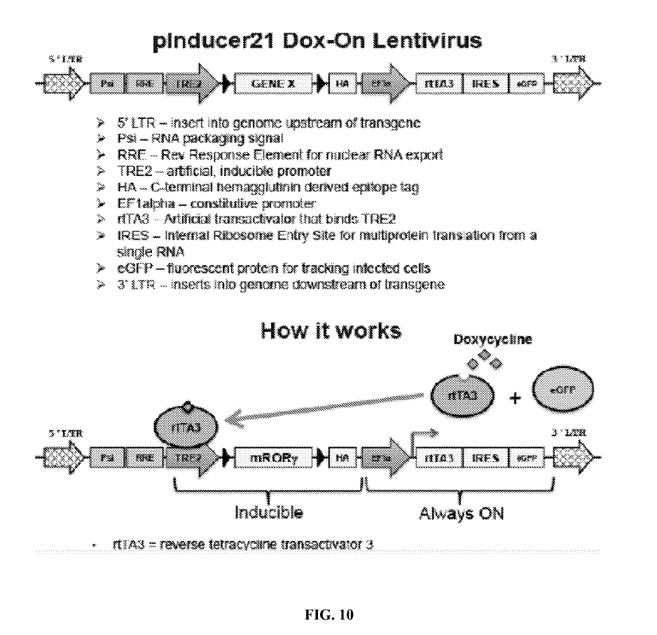

Inducible lentiviral constructs (pinducer21) were used to infect Hoxa9 HF1 myeloid precursor cells. Cells infected with this construct produce mRORgamma upon the addition of doxycycline and the activation of the constitutively expressed rtTA3 transcriptional transactivation protein (FIG. 10).

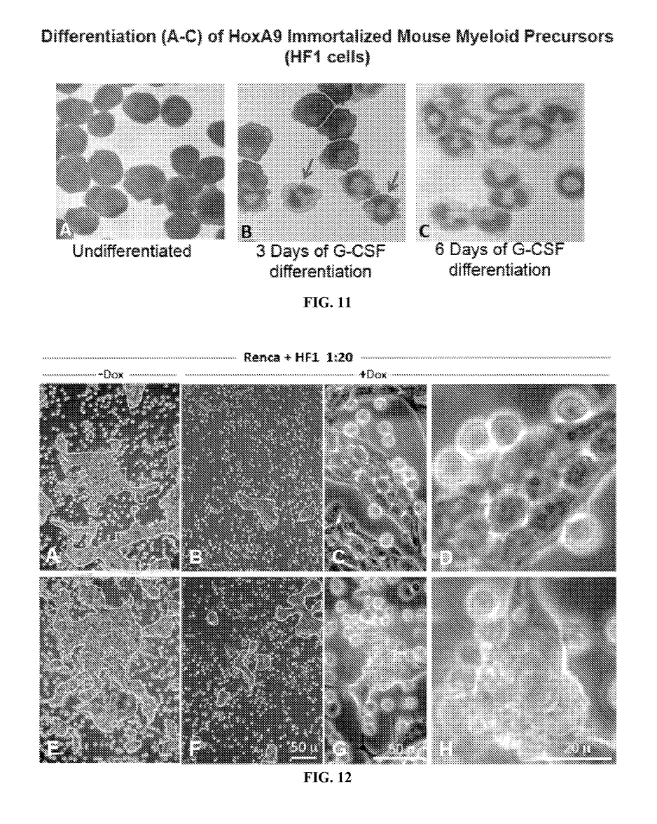

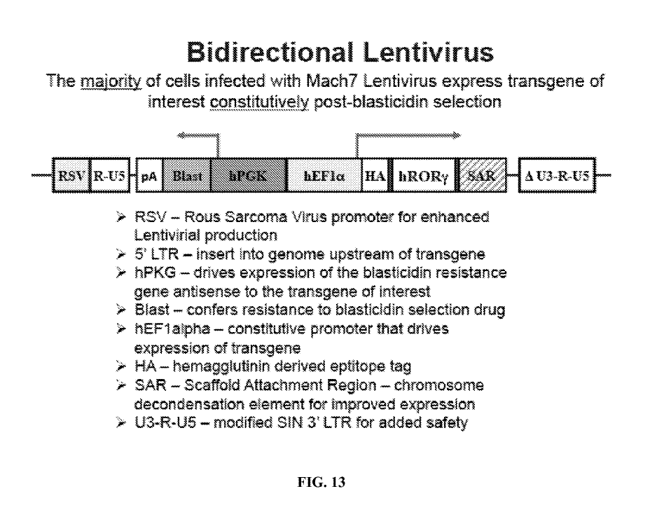

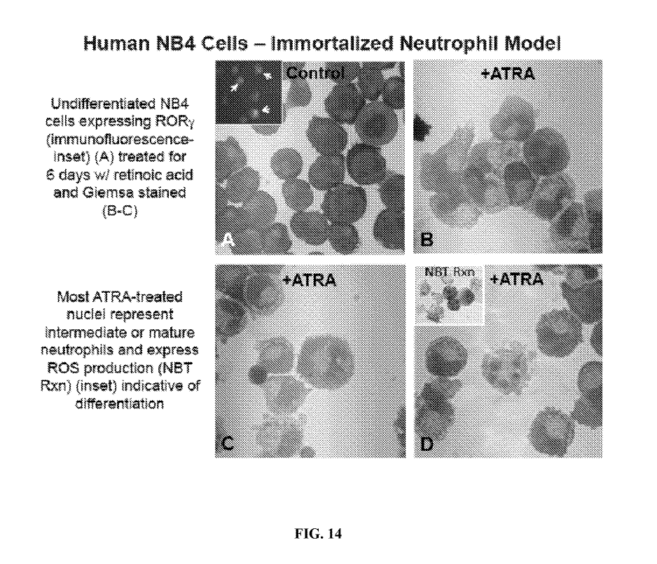

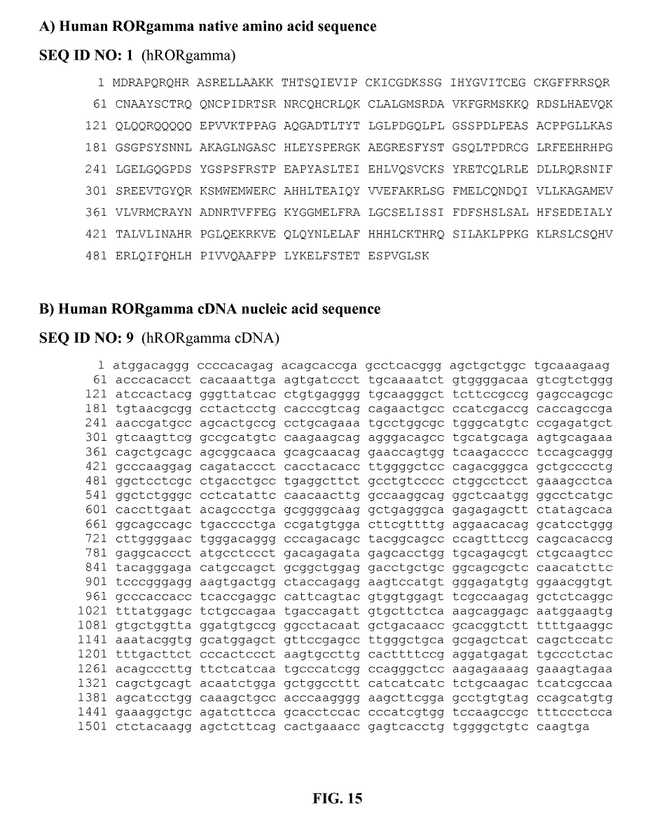

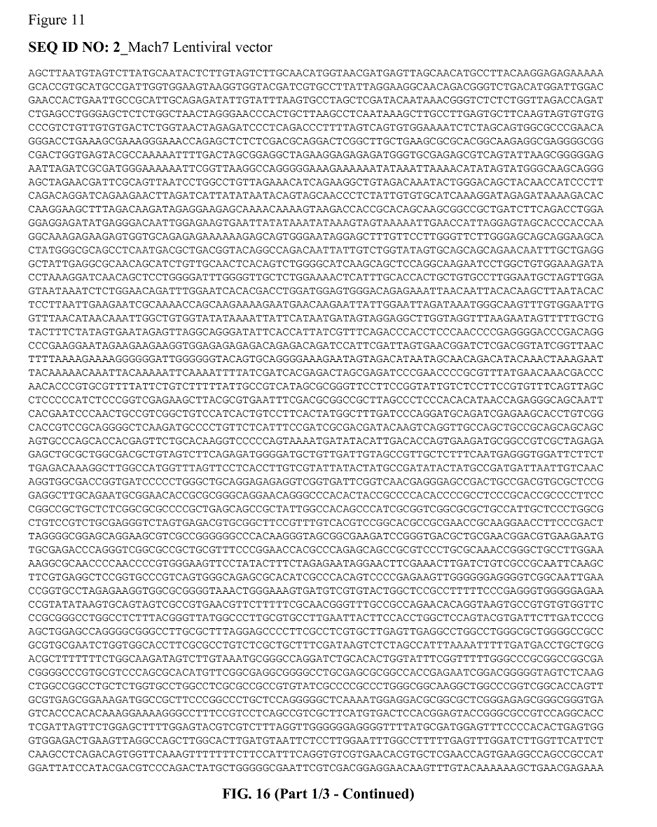

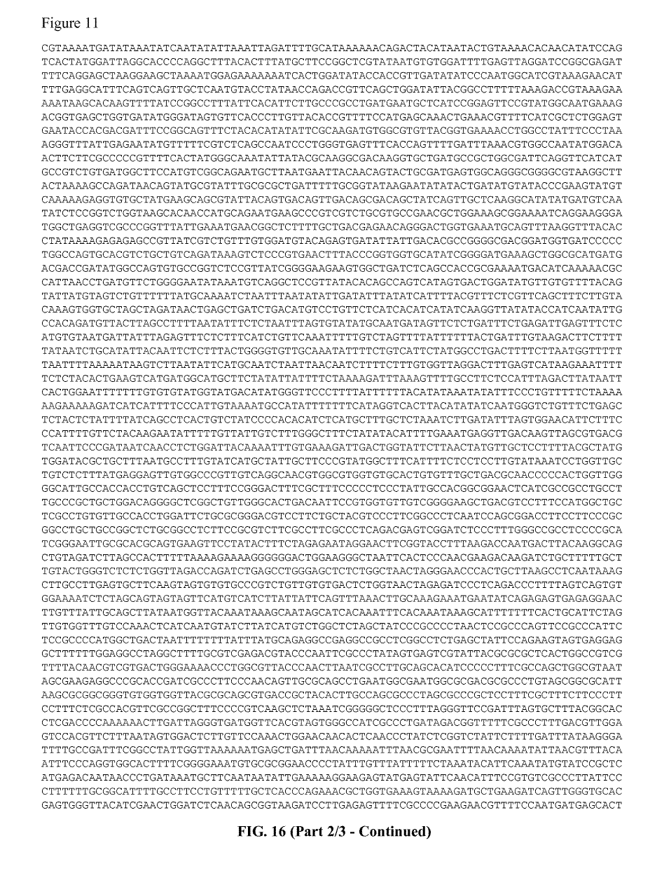

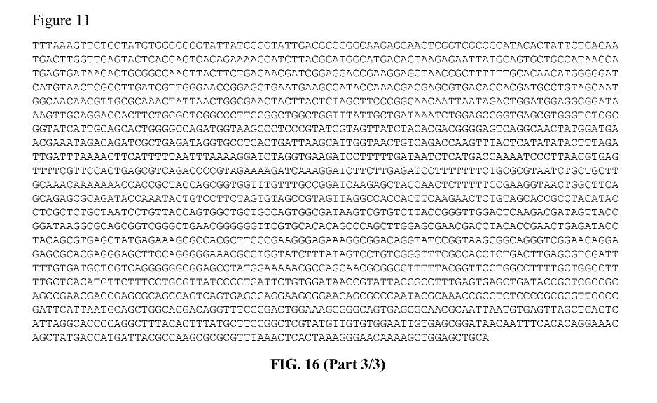

To differentiate the HoxA9 HF1 cell line into neutrophils, HF1 cells were maintained in standard growth media or washed with saline (FIG. 11A) and grown in the presence of 20 ng/mL of G-CSF for three days (FIG. 11B) or for six days (FIG. 11C). Cells were cytospun at indicated times and stained with Giemsa. The arrows shown in FIG. 11B indicate cells that clearly demonstrated multilobed nuclei characteristic of neutrophils.

Immune cells modified with pinducer21 Lentivirus RORgamma were able to reduce the number of cancer cells in an in vitro assay. Renca cells were allowed to adhere and grow overnight to a tissue culture treated 6-well dish for 24 hr. Then, differentiated HF1-inducible RORgamma cells were added at a ratio of 20:1 (neutrophils:Renca) without (minus doxycycline (FIGS. 12A and 12E) or with doxycycline (plus doxycycline FIGS. 12B, 12C, 12D, 12F, 12G, and 12H). After a 48 hr incubation period, media containing neutrophils was aspirated and plates were washed with PBS before fixing Renca cells with 4% paraformaldehyde with PBS. Transgenic HF1 cells that were both differentiated and induced for RORgamma expression via doxycycline consistently cleared more Renca cells than differentiated HF1 transgenic cells in which RORgamma was not induced with doxycycline.

### Example 4: Xenograft Assays in Mice

Xenograft experiments are conducted using athymic C57BL/6 foxn1/foxn1 nude mice, which lack production of mature T cells. Foxn1/foxn1 are also nude resulting from the inability to produce normal hair follicles, this trait allows easier visualization of subcutaneous xenografts. Initially, both Renca and S180 cancer cells serve as a positive control for the assay. Subcutaneous tumors ranging from 400 to 800 mg in size are generated through direct injection of S180 cells harvested from culture. Treatment of mice begin with transgenic RORgamma-MPRO transgenic cells in which cells have been fully differentiated and RORgamma expression has been induced. Several dosages of RORgamma-MPRO derived neutrophils are tested in intraperitoneal injections, with a mean number of 4×10⁸ neutrophils. Mice are monitored daily for changes in tumor volume beginning 24 hrs following the transfer of RORgamma-MPRO cells. Upon confirming an effect of RORgamma-MPRO cells versus uninduced RORgamma-MPRO and parental MPRO controls, tests are immediately conducted with the KLN 205 and LL/2 mouse lung cancer cell before progressing to tumorigenic human cell lines. Mice treated with cancer cell lines that develop tumors >10% of bodyweight are sacrificed and tumors and surrounding tissue fixed on formalin for sectioning and histology. Mice with evidence of tumor regression are kept until regression has become static for >30 days or tumor growth resumes. For tumors fitting either criterion, mice are sacrificed and tumors harvested for pathology. Xenograft assays performed with human cancer cell lines are done in parallel to those done in mice.

### Example 5: Modification of Human Myeloid Precursors Based on Mouse Experiments

In order to deliver a human homolog of RORgamma into human myeloid precursors, sequence verified human RORgamma are cloned into the pinducer21 vector (SEQ ID NO: 7; FIG. 20). The human RORgamma-pinducer21 construct are used to infect human CD34⁺ myeloid precursors that are available commercially from several tissue sources such as cord blood (StemCell Technologies Inc.), bone marrow (Astarte Biologics Inc.), and peripheral blood (Allcells Inc.). Lentiviral supernatant are used to infect newly thawed CD34⁺ myeloid precursors after a 24 hr recovery period in culture media. Lentiviral supernatant are generated using standard methods and then concentrated before infecting CD34⁺ in a small volume for ˜2-4 hours. Both during and after lentiviral infection, CD34⁺ cells are maintained and expanded in specialized media containing SCF, Flt3L, IL-2 and TPO. This specific combination of cytokines and growth factors is known in the art to expand the starting population of CD34⁺ cells an average of 40 fold within 8 days (Murray et al. Exp Hematol. (6):1019-28, 1999; Singh et al. Radiat Res. 177(6):781-91, 2012). Moreover, the majority of cells on day 8 are found to be composed of myeloid progenitors based on the presence of cell surface markers CD33 and CD34.

### Example 6: Differentiation and Expression of Human RORgamma in a Human Model of Neutrophil Differentiation

To deliver human RORgamma into human myeloid precursors, human RORgamma was cloned into the Mach7 consitutive Lentiviral vector (FIG. 13). The Mach7 Lentiviral vector drives expression of human RORgamma (hRORgamma) via the human EF1 alpha promoter.

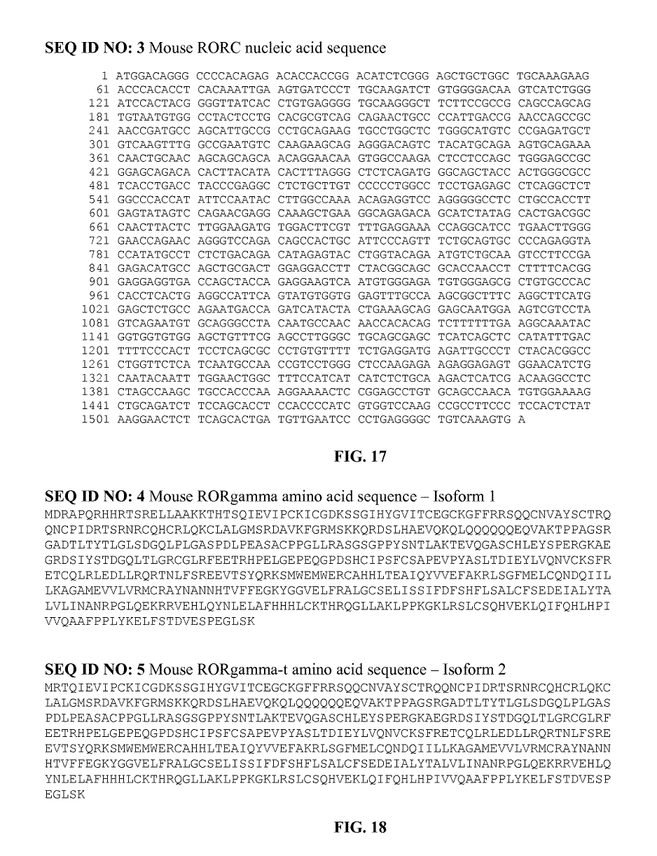

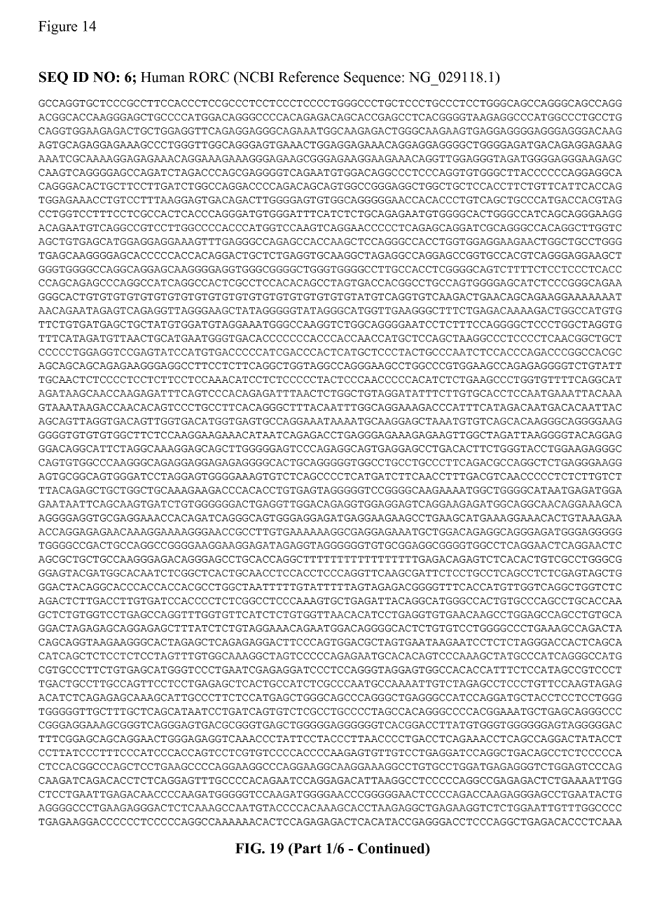

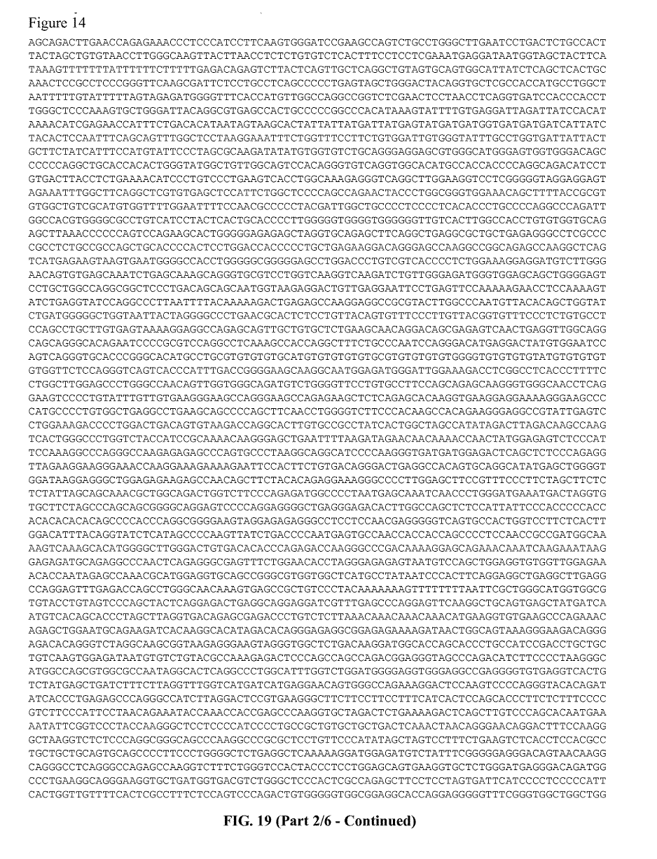

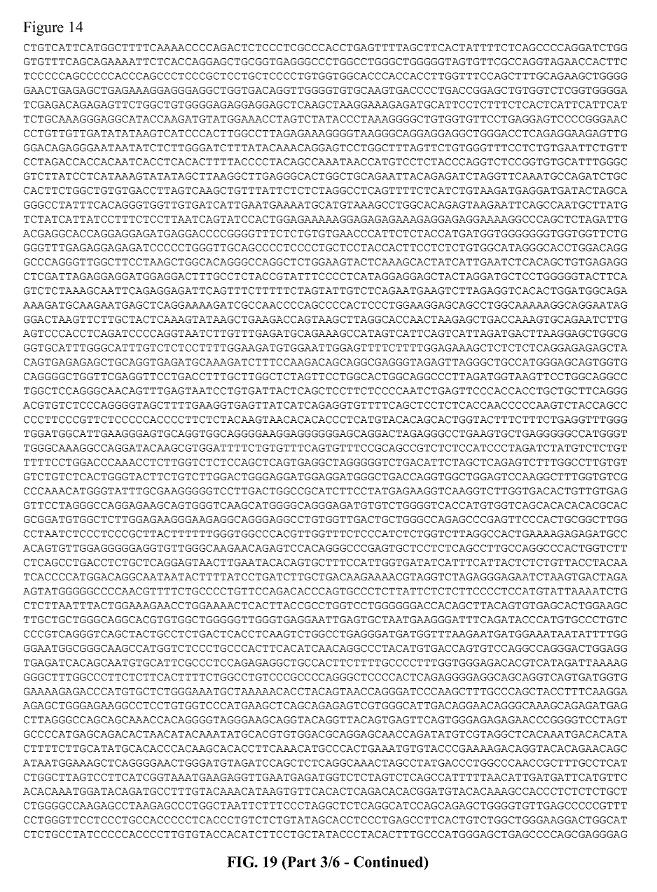

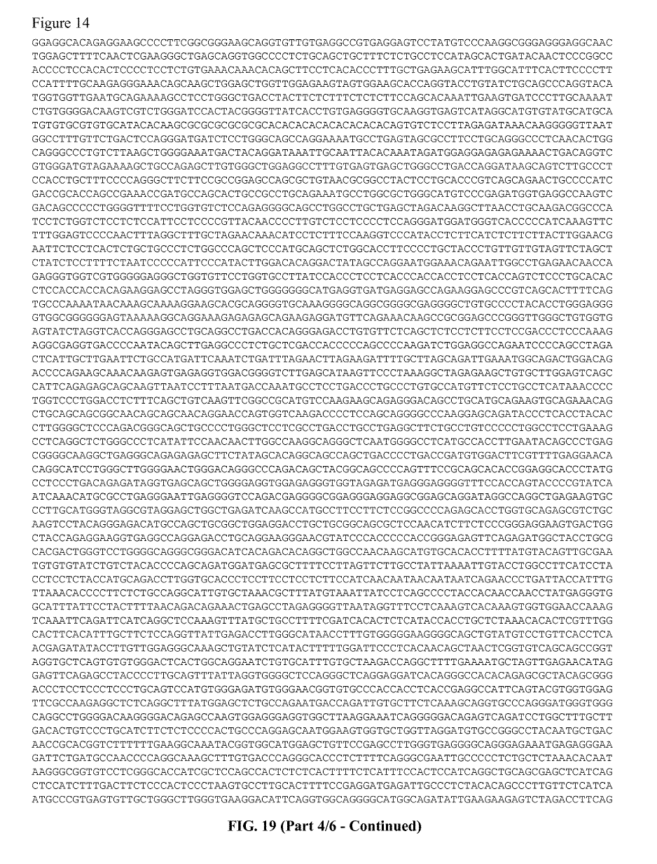

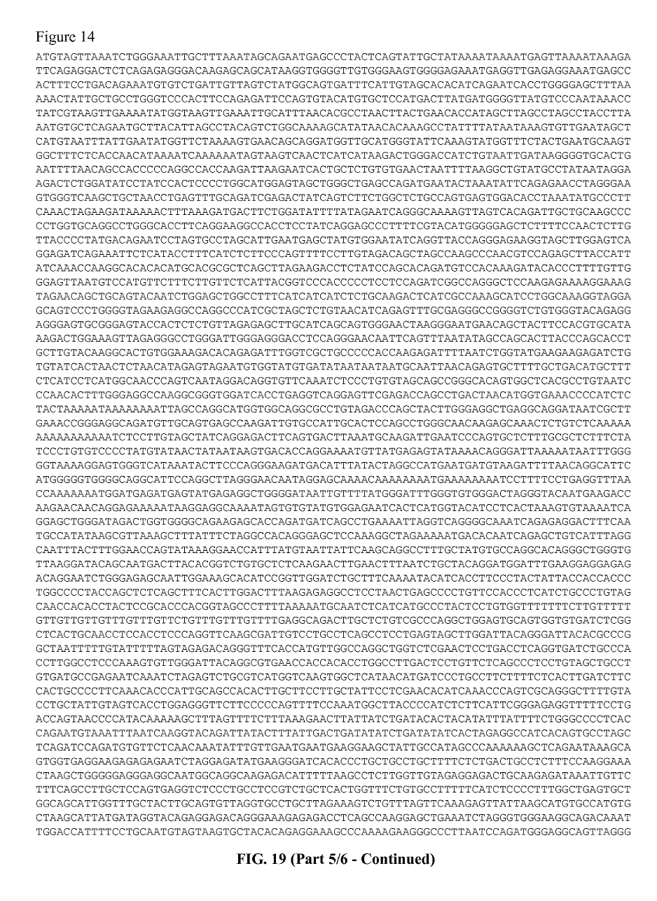

NB4 cells were infected with Mach7-hRORgamma Lentivirus and stained with anti-HA antibody to visualize hRORgamma (FIG. 14A, inset). NB4 cells that survived blasticidin selection were positive for hRORgamma. Cells were further differentiated with all trans retinoic acid (ATRA) and after six days cytospun onto slides and stained with Giemsa (FIGS. 14B-14D). Further, cells at Day 6 were subjected to a nitroblue tetrazolium assay (NBT) in order to test the ability of differentiated cells to reduce nitroblue tetrazolium, a hallmark of differentiated neutrophils. Cells were stimulated with phorbal myristate acetate (PMA) for 30 minutes and subsequently cytospun and fixed with 4% paraformaldehyde. Reduction of NBT resulted in a black precipitate (FIG. 14D, inset) in approximately 10% of cells, as reported in the literature for normal differentiation.

### Example 7: Use of qRT-PCR/RNA-Seq and ChIP-Seq to Determine RORgamma Immune Cell Profile and Regulated Factors

RORgamma immune cell lines that have been confirmed to induce cell death (apoptosis or necrosis) of target cancer cell lines in cell killing assays are utilized for quantitative reverse-transcription PCR (qRT-PCR) as well as deep sequencing experiments. Results allow to determine if the artificially produced, functional RORgamma immune cells mirror their SR/CR counterparts. Specifically, total RNA are isolated from (a) control cells as well as RORgamma transgenic cells, (b) cells in the ‘off’ state as well as those induced for transgene expression with doxycycline and (c) cells that are in the myeloid precursor state and differentiated with ATRA into neutrophils. Total RNA are subjected to Taqman qRT-PCR reactions (Life Technologies) and transcripts previously determined to undergo significant change in levels (+ or −4 fold) are examined for similarities in the artificially generated SR/CR-like myeloid lines. In parallel to RNA-based experiments, RORgamma transgenic MPRO cells and control cell lines are grown up in an uninduced and undifferentiated state, and then induced to differentiate. After several days of induction, when significant (>95%) differentiation has occurred, cells are checked for viability via Trypan Blue exclusion and are used if they show high viability. A total of ˜2×10⁷ cells are harvested for each transgenic line and processed for ChIP-Seq following standard protocols of: cross-linking with formaldehyde, sonication, immunoprecipitation (e.g. pull down of RORgamma with anti-mouse RORgamma antibody), reversal of cross-link and isolation of ChIP'd DNA and finally library construction and HiSeq sequencing. Results obtained from ChIP-Seq are cross referenced to the results from the Taqman assays to establish which direct RORgamma targets are greatly altered in neutrophils with cancer cell killing ability. Based on the RNA-Seq and in silico promoter analysis data, several strong RORgamma regulated gene candidates are selected.

### Example 8: Defining Critical Factors Downstream of RORgamma

To determine which RORgamma targets may be either partially responsible or dispensable for the SR/CR-like phenotype of transgenic myeloid lineage cells, either ectopic expression of cDNAs or shRNA knockdown are used. Ectopic expression of cDNAs of potential importance are tested in two ways. In the first scenario, cDNAs confirmed to be RORgamma targets and significantly upregulated in cells are ectopically expressed in MPRO cells through the use of pinducer21 (particularly, unmodified pinducer21 vector). In the second scenario, cDNAs representing transcripts downregulated in neutrophils expressing RORgamma are reintroduced. Artificially increasing levels of downregulated genes may, in some cases, result in a nullification of the cancer killing or possibly cancer homing capabilities of SR/CR-like neutrophils. The goal is to systematically identify genes downstream of RORgamma whose downregulation is essential for the RORgamma phenotype. For these experiments, pinducer21 is modified by substituting the GFP cassette with a red fluorescent protein (RFP) open reading frame followed by a 2A peptide splice site and puromycin resistance. In doing so, the RORgamma transgenic cells that are already fluorescent for GFP are again modified. In the case of ORFs that have expression upregulated by RORgamma, shRNAs are delivered into transgenic RORgamma-MPRO cells through the use the TRIPZ Lentiviral vector (Thermo Fisher Scientific, Inc.) which marks cells producing the shRNA of interest through the parallel production of RFP. For these experiments the shRNA insert is either engineered into Lentivirus or purchased ready-made TRIPZ viral supernatant. The production of an additive effect in previously unmodified MPRO cells is considered for any proteins having any impact on the effectiveness of RORgamma. For instance, if the knockdown of the highly downregulated CD148 receptor and upregulation of the highly unregulated scavenger receptor 2 (Scart2) produce a phenotypic effect alone, their effects are combined through the combination of pinducer21 (upregulation) and TRIPZ (downregulation) in an off the shelf MPRO cell line.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A composition comprising a viral vector comprising a nucleic acid sequence encoding retinoid-related orphan receptor gamma (RORgamma), wherein expression of RORgamma is under the control of a neutrophil specific promoter.

2. A cell comprising a viral vector comprising a nucleic acid sequence encoding RORgamma, wherein expression of RORgamma is under the control of a neutrophil specific promoter.

3. A CD34 positive (CD34⁺) cell comprising a viral vector comprising a nucleic acid sequence encoding RORgamma, wherein expression of RORgamma is under the control of a promoter selected from the group consisting of a neutrophil specific promoter, a constitutive promoter, and an inducible promoter.

4. A CD34⁺ cell that is committed to differentiate into a neutrophil, the cell comprising a viral vector comprising a nucleic acid sequence encoding RORgamma, wherein expression of RORgamma is under the control of a promoter selected from the group consisting of a neutrophil specific promoter, a constitutive promoter, and an inducible promoter.

5. The composition of claim 1, wherein the viral vector is selected from the group consisting of a lentiviral vector, retroviral vector, adenoviral vector, and adeno-associated viral (AAV) vector.

6. The composition of claim 1, wherein the neutrophil specific promoter is CD11B (SEQ ID NO. 8).

7. A method of treating a cancer in a mammal in need thereof, the method comprising administering to the mammal a composition comprising a viral vector comprising a nucleic acid sequence encoding retinoid-related orphan receptor gamma (RORgamma), wherein expression of RORgamma is under the control of a promoter selected from the group consisting of a neutrophil specific promoter, a constitutive promoter, and an inducible promoter.

8. A method of treating a cancer in a mammal in need thereof, the method comprising administering to the mammal a cell comprising a viral vector comprising a nucleic acid sequence encoding RORgamma, wherein expression of RORgamma is under the control of a promoter selected from the group consisting of a neutrophil specific promoter, a constitutive promoter, and an inducible promoter.

9. A method of treating a cancer in a mammal in need thereof, the method comprising administering to the mammal a CD34 positive (CD34⁺) cell comprising a viral vector comprising a nucleic acid sequence encoding RORgamma, wherein expression of ROR gamma is under the control of a promoter selected from the group consisting of a neutrophil specific promoter, a constitutive promoter, and an inducible promoter.

10. A method of treating a cancer in a mammal in need thereof, the method comprising administering to the mammal a CD34⁺ cell that is committed to differentiate into a neutrophil, the cell comprising a viral vector comprising a nucleic acid sequence encoding RORgamma, wherein expression of RORgamma is under the control of a promoter selected from the group consisting of a neutrophil specific promoter, a constitutive promoter, and an inducible promoter.

11. A method for providing anti-tumor immunity in a mammal, the method comprising administering to the mammal a composition comprising a viral vector comprising a nucleic acid sequence encoding RORgamma, wherein expression of RORgamma is under the control of a promoter selected from the group consisting of a neutrophil specific promoter, a constitutive promoter, and an inducible promoter.

12. A method for stimulating innate immune response to a cell population or tissue in a mammal, the method comprising administering to a mammal a composition comprising a viral vector comprising a nucleic acid sequence encoding RORgamma, wherein expression of RORgamma is under the control of a promoter selected from the group consisting of a neutrophil specific promoter, a constitutive promoter, and an inducible promoter.

13. The method of claim 7, wherein the viral vector is selected from the group consisting of a lentiviral vector, retroviral vector, adenoviral vector, and adeno-associated viral (AAV) vector.

14. The method of claim 7, wherein the neutrophil specific promoter is CD11B (SEQ ID NO. 8).

15. A method of diagnosing the activation or activity of anti-tumor immunity in a mammal in need thereof, the method comprising determining the expression level of retinoid-related orphan receptor gamma (RORgamma) in a biological sample from the mammal, wherein an increase in the expression level of RORgamma as compared with a normal control level of RORgamma expression is an indication that the mammal has or is developing anti-tumor immunity.

16. The method of claim 15, wherein the biological sample comprises at least one selected from the group consisting of blood, white blood cells and neutrophils.

17. The method of claim 15, wherein the expression level is at least 10% greater than the normal control level.

18. The method of claim 15, wherein the expression level is determined by a method selected from the group consisting of detecting mRNA of the gene, detecting a protein encoded by the gene, and detecting a biological activity of the protein encoded by the gene.

19. The method of claim 7, wherein the mammal is a human.

20. A kit comprising probe sets for the retinoid-related orphan receptor gamma (RORgamma) and instructions for use thereof, wherein the instructions comprise:
i. detecting the level of RORgamma in innate immune cells in a sample from a mammal in need thereof;
ii. providing an indication on presence or absence of anti-tumor immunity; and
iii. providing a recommendation of whether or not anti-tumor immunity treatment comprising administering to the mammal a composition comprising a viral a nucleic acid sequence encoding RORgamma, wherein expression of RORgamma is under the control of a promoter selected from the group consisting of a neutrophil specific promoter, a constitutive promoter, and an inducible promoter, should be administered, modified, or terminated in the mammal.

21. The cell of claim 2, wherein the viral vector is selected from the group consisting of a lentiviral vector, retroviral vector, adenoviral vector, and adeno-associated viral (AAV) vector.

22. The composition of claim 2, wherein the neutrophil specific promoter is CD11B (SEQ ID NO. 8).

23. The cell of claim 3, wherein the viral vector is selected from the group consisting of a lentiviral vector, retroviral vector, adenoviral vector, and adeno-associated viral (AAV) vector.

24. The composition of claim 3, wherein the neutrophil specific promoter is CD11B (SEQ ID NO. 8).

25. The cell of claim 4, wherein the viral vector is selected from the group consisting of a lentiviral vector, retroviral vector, adenoviral vector, and adeno-associated viral (AAV) vector.

26. The composition of claim 4, wherein the neutrophil specific promoter is CD11B (SEQ ID NO. 8).

27. The method of claim 8, wherein the viral vector is selected from the group consisting of a lentiviral vector, retroviral vector, adenoviral vector, and adeno-associated viral (AAV) vector.

28. The method of claim 8, wherein the neutrophil specific promoter is CD11B (SEQ ID NO. 8).

29. The method of claim 9, wherein the viral vector is selected from the group consisting of a lentiviral vector, retroviral vector, adenoviral vector, and adeno-associated viral (AAV) vector.

30. The method of claim 9, wherein the neutrophil specific promoter is CD11B (SEQ ID NO. 8).

31. The method of claim 10, wherein the viral vector is selected from the group consisting of a lentiviral vector, retroviral vector, adenoviral vector, and adeno-associated viral (AAV) vector.

32. The method of claim 10, wherein the neutrophil specific promoter is CD11B (SEQ ID NO. 8).

33. The method of claim 11, wherein the viral vector is selected from the group consisting of a lentiviral vector, retroviral vector, adenoviral vector, and adeno-associated viral (AAV) vector.

34. The method of claim 11, wherein the neutrophil specific promoter is CD11B (SEQ ID NO. 8).

35. The method of claim 12, wherein the viral vector is selected from the group consisting of a lentiviral vector, retroviral vector, adenoviral vector, and adeno-associated viral (AAV) vector.

36. The method of claim 12, wherein the neutrophil specific promoter is CD11B (SEQ ID NO. 8).
